(19) United States
(12) Patent Application Publication
(10) Pub. No.: US 2025/0073334 A1
(43) Pub. Date: Mar. 6, 2025

(54) INJECTABLE HYDROGEL FOR SUSTAINED CO-DELIVERY OF AN ANTIGEN AND AN ADJUVANT, AND USES THEREOF

(71) Applicant: The Regents of the University of California, Oakland, CA (US)

(72) Inventors: Szu-Wen Wang, Irvine, CA (US); Lu Wang, Irvine, CA (US); David Huw Davies, Irvine, CA (US); Aaron Ramirez, Irvine, CA (US)

(21) Appl. No.: 18/826,093

(22) Filed: Sep. 5, 2024

Related U.S. Application Data
(60) Provisional application No. 63/536,695, filed on Sep. 5, 2023.

(51) Int. Cl. A61K 39/00; A61K 39/145; A61K 39/215; A61K 39/39

(52) U.S. Cl. CPC A61K 40/45; A61K 39/0002; A61K 39/0011; A61K 39/145; A61K 39/215; A61K 39/39; A61K 2039/54; A61K 2039/55505; A61K 2039/55516; A61K 2039/55555; A61K 2039/55561; A61K 2039/55583; A61K 2039/627

(57) ABSTRACT

The disclosure provides for a vaccine depot formulation that comprises a biodegradable thermosensitive hydrogel that has been loaded or embedded with nanoparticles that comprise an antigen and adjuvant, and uses thereof for protecting a subject from an infection or disease.

### CROSS REFERENCE TO RELATED APPLICATIONS

This application claims priority under 35 U.S.C. § 119 from Provisional Application Ser. No. 63/536,695, filed Sep. 5, 2023 the disclosure of which is incorporated herein by reference.

### STATEMENT OF GOVERNMENT SUPPORT

This invention was made with Government support under Grant No. HDTRA1-1-18-0036, awarded by the Department of Defense/Defense Threat Reduction Agency. The Government has certain rights in the invention.

### INCORPORATION BY REFERENCE OF SEQUENCE LISTING

Accompanying this filing is a Sequence Listing entitled, “00058-080001.xml” created on Sep. 5, 2024 and having 56.2 KB of data, machine formatted on IBM-PC, MS-Windows operating system. The sequence listing is hereby incorporated by reference in its entirety for all purposes.

### TECHNICAL FIELD

The disclosure provides for a vaccine depot formulation that comprises a biodegradable thermosensitive hydrogel that has been loaded or embedded with nanoparticles that comprise an antigen and adjuvant and uses thereof for protecting a subject from an infection or disease.

### BACKGROUND

Vaccines have been very effective at protecting against infectious diseases that have historically posed serious threats to human health. However, for some pathogens such as SARS-CoV-2 or influenza, emergence of new variants due to antigenic drift and the lack of durability in the immune response can lead to low vaccine effectiveness and the need for multiple booster immunizations. However, multiple immunization regimens are costly and inconvenient, and undesirable for the patients, as seen in the COVID-19 pandemic. Therefore, there is an important need to investigate vaccine design and delivery strategies which yield broader, more durable protection. Recent studies have shown that vaccine release kinetics can be important in establishing lasting and efficacious immunity. For example, slowing the release of HIV vaccines can result in higher antibody titers and increased diversity in neutralizing antibodies that target a more diverse set of epitopes, relative to immune responses from conventional bolus vaccination. This was presumably by allowing rare neutralizing antibody precursors to be recruited into the B cell response. However, in this case, the slow delivery of vaccine was approached by applying multiple small doses, implanting an osmotic pump, or using a microneedle array, which results in multiple doses or higher device cost.

### SUMMARY

Although conventional vaccines are delivered as a solution-based bolus, studies have shown that the rate at which vaccines are exposed to the immune system can affect the strength and breadth of its protection. Accordingly, slow delivery of antigen has great potential to enhance the production of neutralizing antibodies and stimulate the germinal center (GC) response. Provided herein is the development of a vaccine delivery platform which comprises two components: (1) a nanoparticle-conjugated antigen, which can deliver antigen and adjuvant simultaneously and enhance the uptake and activation of dendritic cell; and (2) a thermal-sensitive hydrogel that gels at body temperature, which functions as depot for vaccine and provide long-lasting delivery up to 8 weeks. This platform significantly increased IgG, IgG1, and IgG2c for the model antigen. The immune response data also suggest that both NP and hydrogel plays an important role in this platform, more specifically, conjugating antigen on the nanoparticle increases the Th1 polarized response, and the hydrogel enhanced both Th1 and Th2 responses.

As shown in the studies presented herein, the vaccine delivery platform of the disclosure significantly improved durability (the length of time of the immune response) and increased cross-reactivity between pathogenic (e.g., viral) variants. The results are significant because it decreases the need for multiple booster shots and the ongoing need to predict future pathogenic strains for annual vaccines. For example, the current COVID-19 pandemic has shown the ease at which viruses can mutate. This controlled-release strategy of antigens disclosed herein can be applied towards pathogenic targets that change over time, for example influenza and SARS-CoV-2. Due to the modular design of the vaccine delivery platform of the disclosure, the successful implementation of this strategy could have broader applicability towards the development of vaccines for other infectious pathogens prone to antigen drifting (e.g., influenza).

In a particular embodiment, the disclosure provides a vaccine depot formulation that provides for the simultaneous delivery of an antigen and an adjuvant, comprising: a biodegradable thermosensitive hydrogel that has been loaded or embedded with nanoparticles that comprise the antigen and the adjuvant, wherein the nanoparticles are hollow and comprise an outer surface and an accessible inner cavity, where the antigen and/or the adjuvant is conjugated to the outer surface of the nanoparticles, and/or where the antigen and/or the adjuvant is conjugated or loaded into the inner cavity of the nanoparticles. In another embodiment, the antigen is designed to promote a targeted immune response when the vaccine depot is administered in vivo. In a further embodiment, the vaccine depot formulation is suitable for injection by the biodegradable thermosensitive hydrogel being in a liquid state at normal ambient temperatures but solidifying into a gel-like state at a normal body temperature. In yet a further embodiment, the biodegradable thermosensitive hydrogel is in a liquid state at a temperature less than 30° C. In another embodiment, the biodegradable thermosensitive hydrogel is selected from poloxamer-407/188, poloxamer-407, chitosan, poloxamer-chitosan, cellulose, methylcellulose, sodium carboxymethyl cellulose, PLGA-PEG-PLGA (PPP), PCL-PEG-PCL, and poly(N-isopropylacrylamide). In yet another embodiment, the biodegradable thermosensitive hydrogel is PPP. In a further embodiment, the antigen is a foreign antigen from a pathogen selected from a bacterium, a fungus or a virus. In yet a further embodiment, the bacterium is selected from Actinomyces israelii, Bacillus anthracis, Bacillus cereus, Bartonella henselae, Bartonella quintana, Bordetella pertussis, Borrelia burgdorferi, Borrelia garinii, Borrelia afzelii, Borrelia recurrentis, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diphtheriae, Enterococcus faecalis, Enterococcus faecium, Escherichia coli, Francisella tularensis, Haemophilus influenzae, Helicobacter pylori, Legionella pneumophila, Leptospira interrogans, Leptospira santarosai, Leptospira weilii, Leptospira noguchii, Listeria monocytogenes, Mycobacterium leprae, Mycobacterium tuberculosis, Mycobacterium ulcerans, Mycoplasma pneumoniae, Neisseria gonorrhoeae, Neisseria meningitidis, Pseudomonas aeruginosa, Rickettsia rickettsia, Salmonella typhi, Salmonella typhimurium, Shigella sonnei, Staphylococcus aureus, Staphylococcus epidermidis, Staphylococcus saprophyticus, Streptococcus agalactiae, Streptococcus pneumoniae, Streptococcus pyogenes, Treponema pallidum, Ureaplasma urealyticum, Vibrio cholerae, Yersinia pestis, Yersinia enterocolitica, and Yersinia pseudotuberculosis. In another embodiment, the fungus is selected from Absidia corymbifera, Absidia ramose, Achorion gallinae, Actinomadura spp., Ajellomyces dermatididis, Aleurisma brasiliensis, Allersheria boydii, Arthroderma spp., Aspergillus flavus, Aspergillus fumigatu, Basidiobolus spp, Blastomyces spp, Cadophora spp, Candida albicans, Cercospora apii, Chrysosporium spp, Cladosporium spp, Cladothrix asteroids, Coccidioides immitis, Cryptococcus albidus, Cryptococcus gattii, Cryptococcus laurentii, Cryptococcus neoformans, Cunninghamella elegans, Dematium wernecke, Discomyces israelii, Emmonsia spp, Emmonsiella capsulate, Endomyces geotrichum, Entomophthora coronate, Epidermophyton floccosum, Filobasidiella neoformans, Fonsecaea spp., Geotrichum candidum, Glenospora khartoumensis, Gymnoascus gypseus, Haplosporangium parvum, Histoplasma, Histoplasma capsulatum, Hormiscium dermatididis, Hormodendrum spp., Keratinomyces spp, Langeronia soudanense, Leptosphaeria senegalensis, Lichtheimia corymbifera, Lobmyces loboi, Loboa loboi, Lobomycosis, Madurella spp., Malassezia furfur, Micrococcus pelletieri, Microsporum spp, Monilia spp., Mucor spp., Mycobacterium tuberculosis, Nannizzia spp., Neotestudina rosatii, Nocardia spp., Oidium albicans, Oospora lactis, Paracoccidioides brasiliensis, Petriellidium boydii, Phialophora spp., Piedraia hortae, Pityrosporum furfur, Pneumocystis jirovecii (or Pneumocystis carinii), Pullularia gougerotii, Pyrenochaeta romeroi, Rhinosporidium seeberi, Sabouraudites (Microsporum), Sartorya fumigate, Sepedonium, Sporotrichum spp., Stachybotrys, Stachybotrys chartarum, Streptomyce spp., Tinea spp., Torula spp, Trichophyton spp, Trichosporon spp, and Zopfia rosatii. In yet another embodiment, the virus is selected from Human coronavirus, Human papillomavirus, Torque teno virus, Barmah forest virus, Chikungunya virus, Eastern equine encephalitis virus, Mayaro virus, O'nyong-nyong virus, Ross river virus, Sagiyama virus, Semliki forest virus, Sindbis virus, Venezuelan equine encephalitis virus, Western equine encephalitis virus, Junin arenavirus, Lassa virus, Lymphocytic choriomeningitis virus, Machupo virus, Pichinde virus, Human SARS coronavirus, MERS coronavirus, SARS coronavirus, Encephalomyocarditis virus, Cosavirus A, Human cytomegalovirus, Human T-lymphotropic virus, Hepatitis delta virus, Adeno-associated virus, Ebolavirus, Human rhinovirus, Coxsackievirus, Echovirus, Human enterovirus, Poliovirus, Human parvovirus B19, Murray valley encephalitis virus, Dengue virus, Japanese encephalitis virus, Langat virus, Louping ill virus, St. louis encephalitis virus, Tick-borne powassan virus, West Nile virus, Yellow fever virus, Zika virus, Hantaan virus, New York virus, Puumala virus, Seoul virus, Hendra virus, Nipah virus, Hepatitis virus, Influenza virus, Aichi virus, Human immunodeficiency virus, Cercopithecine herpesvirus, Epstein-Barr virus, Australian bat lyssavirus, Duvenhage virus, Lagos bat virus, Mokola virus, Rabies virus, European bat lyssavirus, Human astrovirus, Lake Victoria marburgvirus, Human adenovirus, Molluscum contagiosum virus, Measles virus, Human papillomavirus, Crimean-Congo hemorrhagic fever virus, Dugbe virus, Norwalk virus, Southampton virus, Oropouche virus, Bunyamwera virus, Bunyavirus La Crosse, Bunyavirus snowshoe hare, Hepatitis B virus, Human respiratory syncytial virus, Monkeypox virus, Cowpox virus, Horsepox virus, Vaccinia virus, Variola virus, Yaba monkey tumor virus, Yaba-like disease virus, Orf virus, GB virus C/Hepatitis G virus, Punta toro phlebovirus, Rift valley fever virus, Sandfly fever Naples phlebovirus (Toscana virus), Sandfly fever sicilian virus, Uukuniemi virus, BK polyomavirus, JC polyomavirus, KI Polyomavirus, Merkel cell polyomavirus, WU polyomavirus, Human parainfluenza, Rosavirus, Human herpesvirus, Rotavirus, Rubella virus, Mammalian orthorubulavirus (Simian virus), Mumps virus, Salivirus A, Sapporo virus, Banna virus, Eastern chimpanzee simian foamy virus, Simian foamy virus, Dhori virus, Vientovirus, Human torovirus, Varicella-zoster virus, Chandipura virus, Isfahan virus, and Vesicular stomatitis virus. In a further embodiment, the antigen is from an influenza virus, SARS-CoV-2, or Coxiella burnetii. In yet a further embodiment, the antigen is an antigenic protein, or antigenic peptide. In another embodiment, the antigen is an endogenous antigen from a cancer or is associated with an immune disease. In yet another embodiment, the antigen is a cancer tumor-specific antigen selected from tumor-specific antigen selected from NY-ESO-1, gp100, CT26, alphafetoprotein (AFP), carcinoembryonic antigen (CEA), CA-125, CA15-3, CA19-9, MUC-1, epithelial tumor antigen (ETA), tyrosinase, melanoma-associated antigen (MAGE), abnormal products of ras or p53, CTAG1B, MAGEA1, and HER2/neu. In a further embodiment, the nanoparticles are hollow protein nanoparticles. In yet a further embodiment, the hollow protein nanoparticles are based on ferritin, TIP60, or hepatitis B virus (HBV) surface-antigen protein. In a further embodiment, the hollow protein nanoparticles are based on the E2 subunit, or a portion thereof, of the pyruvate dehydrogenase complex (PDC) from the Geobacillus stearothermophilus. In yet another embodiment, the lipoyl domain of the E2 subunit has been removed resulting in a E2 protein that comprises 256 amino acids having the sequence of SEQ ID NO:44. In a further embodiment, the E2 subunit of the PDC has been recombinantly modified to substitute one or more amino acids with cysteines. In a further embodiment, the recombinant E2 subunit has a polypeptide sequence that is at least 95%, at least 98%, or at least 99% identical to the sequence presented in SEQ ID NO:5 and which has an amino acid substitution of D214C in comparison to SEQ ID NO:45. In a certain embodiment, the recombinant E2 subunit has a polypeptide sequence of SEQ ID NO:5, SEQ ID NO:7, or SEQ ID NO:9. In a further embodiment, the recombinant E2 subunit has a polypeptide sequence of SEQ ID NO:5. In yet a further embodiment, the adjuvant is selected from ssRNA, aluminum hydroxide, aluminum phosphate, and aluminum potassium sulfate, MGN1703, AS04, MF59, and AS01_{B}. In another embodiment, the adjuvant is an CpG oligodeoxynucleotide based adjuvant. In yet another embodiment, the CpG oligodeoxynucleotide based adjuvant is selected from CpG1018, CpG1826, CpG ODN 1466, CpG ODN PB3, CpG ODN BW005, CpG Alum, CPG 21424, CpG 7909, CpG-ODN 2135, ODN K3, CpG ODN 10101, CpG-28, CpG ODN C274, CpG ODN C695, CpG ODN #17, CpG-ODN 2722, CpG 8916, CpG 8954, CpG ODN 678, CpG ODN BW015, CpG ODN 658, CpG ODN 640, CpG ODN PB9, CpG ODN BW004, CpG ODN BW103, CpG ODN 110, CpG ODN BW206, CpG ODN 607, CpG ODN 647, CpG ODN 111, CpG ODN 109, CpG ODN 656, CpG ODN 664, CpG ODN C9, CPG2429, CPG5475, CPG21608, CPG21797, CPG21796, CPG21799, CPG21800, CPG21802, CPG21889, CPG23409, CpG-c41, CPG23410, CPG23411, CPG23412, CPG23413, CPG23617, CPG23414, CPG 1681, CPG 2143, CPG 21425, and CPG 21426. In another embodiment, the CpG oligodeoxynucleotide based adjuvant has the sequence of SEQ ID NO:40. In a further embodiment, the antigen is conjugated to the outer surface of the nanoparticles, and wherein the adjuvant is conjugated or loaded into the inner cavity of the nanoparticles. In yet a further embodiment, the adjuvant is conjugated to the outer surface of the nanoparticles, and wherein the antigen is conjugated or loaded into the inner cavity of the nanoparticles. In a certain embodiment, the antigen and/or the adjuvant are conjugated to the outer surface and an accessible inner cavity of the nanoparticles by use of mal-tNTA-Ni, sulfo-SMCC, sortase A ligation, or by use of SpyCatcher/SpyTag. In another embodiment, the antigen is conjugated to the outer surface of the nanoparticles by use of SpyCatcher/SpyTag, and wherein the adjuvant is loaded or conjugated to the inner cavity of the nanoparticles by use of a linker that binds to both the adjuvant and free cysteine groups in the inner cavity of the nanoparticles. In yet another embodiment, the adjuvant has been modified to comprise a terminal aldehyde or benzaldehyde group, and wherein the linker is a N-(β-maleimidopropionic acid) hydrazide (BMPH) linker. In a further embodiment, the adjuvant has the sequence of SEQ ID NO:40 that comprises a terminal 5′ benzaldehyde. In yet a further embodiment, the outer surface of the nanoparticles is conjugated with a SpyTag peptide, and wherein the antigen is conjugated with a SpyCatcher peptide. In a certain embodiment, the SpyTag peptide has the sequence of SEQ ID NO:17, and wherein the SpyCatcher peptide has the sequence of SEQ ID NO: 19. In a further embodiment, the nanoparticles comprise a recombinant E2 subunit that has a polypeptide sequence that is at least 95%, at least 98%, or at least 99% identical to the sequence presented in SEQ ID NO:5 and which has an amino acid substitution of D214C in comparison to SEQ ID NO:45. In a further embodiment, the SpyTag conjugated recombinant E2 protein nanoparticles (ST-E2) have the sequence of SEQ ID NO:21. In yet a further embodiment, the antigen that is conjugated with a SpyCatcher peptide is CBU1910 and the construct (SC-CBU1910) has the sequence of SEQ ID NO:23. In another embodiment, the antigen that is conjugated with a SpyCatcher peptide is H5 hemagglutinin and the construct (SC-H5) has the sequence of SEQ ID NO:43.

In a particular embodiment, the disclosure also provides a method of immunizing a subject comprising: administering one or more doses of the vaccine depot formulation of the disclosure to protect a subject from an infection by a pathogen and/or from a disease. In a further embodiment, the infection and/or disease is selected from Q fever, avian influenza, and SARS-CoV-2. In yet a further embodiment, the vaccine depot formulation is formulated for intramuscular administration, subcutaneous administration, or intravenous administration. In another embodiment, the vaccine depot formulation provides long-lasting, sustained delivery of the antigen for at least 2, 4, 6, 8, 10, or 12 weeks in vivo.

In a certain embodiment, the disclosure also provides a method to make the vaccine depot formulation of the disclosure, comprising: reacting SpyTag conjugated recombinant E2 protein (ST-E2) nanoparticles comprising a plurality of free cysteine groups with a linker and adjuvant comprising a terminal aldehyde or benzaldehyde group, to form ST-E2 nanoparticles comprising the adjuvant; and reacting the ST-E2 nanoparticles comprising the adjuvant with a SpyCatcher conjugated antigen to form the vaccine depot formulation. In a further embodiment, the ST-E2 nanoparticles have the sequence of SEQ ID NO:21. In yet a further embodiment, the linker is a N-(β-maleimidopropionic acid) hydrazide (BMPH) linker. In another embodiment, the adjuvant has the sequence of SEQ ID NO:40 that comprises a terminal 5′ benzaldehyde. In yet a further embodiment, the SpyCatcher conjugated antigen comprises a foreign antigen from a pathogen or an endogenous antigen from a cancer or is associated with an immune disease. In another embodiment, a SpyCatcher peptide that has the sequence of SEQ ID NO:19 is conjugated to the antigen.

In a certain embodiment, the disclosure further provides a vaccine depot formulation as substantially described and/or presented herein.

### DESCRIPTION OF DRAWINGS

FIG. 1 provides a schematic of a hydrogel depot for extended delivery of nanoparticle-based vaccine.

FIG. 2 presents a schematic of hydrogel depot gelation at body temperature. The hydrogel depot material can be easily prepared by dissolving the block polymer in PBS. It is a liquid at room temperature and can be loaded with the antigen and adjuvants by simply mixing. It is injectable under room temperature. After injected and temperature rise above 32.6° C., it turns into a hydrogel.

FIG. 3 presents a schematic of Ag-CpG-E2 nanoparticle conjugation. ST-E2 nanoparticles with 60 SpyTag on the exterior and 60 cysteine groups in the interior are conjugated with aldehyde-modified CpG1826 and SpyCatcher-fused antigen.

FIG. 4 provides for characterization of the conjugated Ag-CpG-E2 nanoparticles. The conjugated nanoparticles were characterized with SDS-PAGE and the amount of CpG and antigen on each nanoparticle was quantified. On each E2 nanoparticle, there are 20.5±1.5 CpG molecules on internal cavity and 24±2 antigens on the surface.

FIG. 5 presents the in vivo release profile of a hydrogel embedded with Ag-CpG-E2 nanoparticles. An in vivo release test was performed in mice to examine the retention time of antigen in the injection site when antigens are embedded in a hydrogel matrix or without hydrogel matrix. Fluorescently labeled E2 NPs were solubilized in PBS or pre-gel polymer solution and injected subcutaneously to the lower back of mice. The total radiant efficiency was monitored with IVIS. After a burst release on day 1, the gel group had about 40 percent of E2 left in the injection site and the release continued until week 8, whereas the bolus group finished releasing on day 4.

FIG. 6 demonstrates the immune response in mice when immunized with s hydrogel embedded with Ag-CpG-E2 nanoparticles. Mice immunized with hydrogel embedded with Ag-CpG-E2 nanoparticles elicited a significantly higher IgG, IgG1, and IgG2c than the control groups with free antigen, the free antigen in hydrogel, and the Ag-CpG-E2 without hydrogel. Up to week 16 after vaccination, antigen-specific IgG of Ag-CpG-E2/Gel group is still higher than the control groups, which shows a great potential of this vaccine to provide a long-term protection. The Ag-CpG-E2 with hydrogel group also showed a more balanced IgG1/IgG2c ratio when compared with the control groups.

FIG. 7A-D provides for nanoparticle synthesis and characterization. (A) Schematic of nanoparticle synthesis with adjuvant CpG and the immunodominant influenza antigen hemagglutinin (H5). CpG was conjugated to the interior cavity of STE2 with BMPH linker followed by the conjugation of SC-H5 onto the exterior of E2 with the ST/SC reaction. (B) SDS-PAGE of the H5-CpG-E2 nanoparticle showing conjugation of CpG and SC-H5, MW_{ST-E2}=30.2 kDa, MW_{ST-E2-CpG}=36.8 kDa, MW_{SC-H5}=71.4 kDa, MW_{H5-E2}=101.6 kDa, MW_{H5-CpG-E2}=108.2 kDa. (C) Hydrodynamic diameters of ST-E2, H5-E2 and H5-CpG-E2 NPs showing physical stability and monodispersity of nanoparticles and shifts in size upon conjugation with adjuvants. (D) Representative TEM image of the H5-CpG-E2 nanoparticles.

FIG. 8 demonstrates that the PPP hydrogel can be used as a depot material to release protein antigens. In vivo release profiles of fluorescently labeled antigen H5 from the injection site for bolus control and from the PPP hydrogel depot. The percentage of signal intensity normalized to 0-time signal intensity is plotted after the background signal is subtracted. n=2 for H5-DL755 group, n=3 for H5-DL755/PPP group.

FIG. 9A-E shows the antibody response elicited by H5-CpG-E2 loaded hydrogel vaccine against the vaccinated H5 variant. (A) Table describing each formulation and its individual components. (B) Immunization schedule and challenge timeline. (C) H5-specific IgG response in serum at week 6. This response is specific to antigen used in the vaccination (H5 variant A/Vietnam 1194/2004). (D) H5-specific IgG response in serum at weeks 2, 6, and 10. This response is specific to antigen used in the vaccination (H5 variant A/Vietnam 1194/2004). (E) H5-specific IgG1/IgG2c response on week 6 (H5 variant A/Vietnam 1194/2004). Data in panel C, D and E are presented as an average ±SEM of 5 individual mice (n=5). Statistical significance was determined by one-way ANOVA test followed by a Tukey multiple comparisons test. *p<0.05, **0.01<p<0.001, ***0.001<p<0.0001, ****p<0.0001.

FIG. 10A-B presents the antibody response elicited by H5-CpG-E2 loaded hydrogel vaccine against 28 different H5 variants. (A) Homosubtypic IgG response to 28 different variants of H5, from week 6 serum. Each column of spots corresponds to the antibody response to a unique H5 antigen variant, with each spot being an average response from n=5 mice. (B) Box plot of homosubtypic IgG response to H5 variants at week 6. Each spot corresponds to response to a different H5 variant (n=5 mice). Plotted in average ±SEM of 28 variants. Statistical significance was determined by one-way ANOVA followed by a Tukey multiple comparisons test. *0.01<p<0.05, **0.01<p<0.001, ***0.001<p<0.0001, ****p<0.0001.

FIG. 11A-B shows that immunization with H5-E2-CpG loaded hydrogel protects mice from the lethal challenge of influenza and improves morbidity. (A) Survival curves, after challenge with H5N1 influenza on week 16 of immunization. Mantel-Cox log-rank test used for survival curve analysis. *p<0.05, **p<0.005, ***p<0.0005. (B) Morbidity plot showing maximal weight loss of each mouse after viral challenge. Data in each group reflects n≥4 individual mice. Statistical significance was determined by one-way ANOVA test followed by a Tukey multiple comparisons test. *p<0.05, **0.01<p<0.001, ***0.001<p<0.0001, ****p<0.0001.

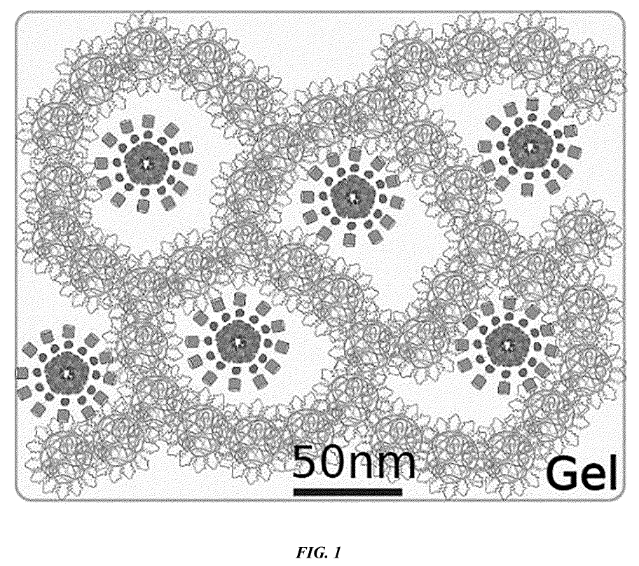

### DETAILED DESCRIPTION

As used herein and in the appended claims, the singular forms “a”, “an”, and “the” include plural referents unless the context clearly dictates otherwise. Thus, for example, reference to “a hydrogel” includes a plurality of such hydrogels and reference to “the adjuvant” includes reference to one or more adjuvants and equivalents thereof known to those skilled in the art, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Although many methods and reagents are similar or equivalent to those described herein, the exemplary methods and materials are disclosed herein.

All publications mentioned herein are incorporated by reference in full for the purpose of describing and disclosing methodologies that might be used in connection with the description herein. The publications are provided solely for their disclosure prior to the filing date of the present application. Nothing herein is to be construed as an admission that the inventors are not entitled to antedate such disclosure by virtue of prior disclosure. Moreover, with respect to any term that is presented in one or more publications that is similar to, or identical with, a term that has been expressly defined in this disclosure, the definition of the term as expressly provided in this disclosure will control in all respects.

It should be understood that this disclosure is not limited to the particular methodology, protocols, and reagents, etc., described herein and as such may vary. The terminology used herein is for the purpose of describing particular embodiments only and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used to described the present invention, in connection with percentages means±1%.

As used herein, “reference sequence” refers to a defined sequence used as a basis for a sequence comparison. A reference sequence may be a subset of a larger sequence, for example, a segment of a full-length gene or polypeptide sequence. Generally, a reference sequence can be at least 20 nucleotide or amino acid residues in length, at least 25 nucleotide or residues in length, at least 50 nucleotides or residues in length, or the full length of the nucleic acid or polypeptide. Since two polynucleotides or polypeptides may each comprise a sequence (i.e., a portion of the complete sequence) that is similar between the two sequences and may further comprise a sequence that is divergent between the two sequences, sequence comparisons between two (or more) polynucleotides or polypeptides are typically performed by comparing sequences of the two polynucleotides or polypeptides over a “comparison window” to identify and compare local regions of sequence similarity.

As used herein, “sequence identity” means that two polypeptide sequences are substantially identical (i.e., on an amino acid-by-amino acid basis) over a window of comparison. The term “sequence similarity” refers to similar amino acids that share the same biophysical characteristics. The term “percentage of sequence identity” or “percentage of sequence similarity” is calculated by comparing two optimally aligned sequences over the window of comparison, determining the number of positions at which the identical residues (or similar residues) occur in both polypeptide sequences to yield the number of matched positions, dividing the number of matched positions by the total number of positions in the window of comparison (i.e., the window size), and multiplying the result by 100 to yield the percentage of sequence identity (or percentage of sequence similarity). Regarding polynucleotide sequences, the terms sequence identity and sequence similarity have comparable meaning as described for protein sequences, with the term “percentage of sequence identity” indicating that two polynucleotide sequences are identical (on a nucleotide-by-nucleotide basis) over a window of comparison. As such, a percentage of polynucleotide sequence identity (or percentage of polynucleotide sequence similarity, e.g., for silent mutations or other mutations, based upon the analysis algorithm) also can be calculated. Maximum correspondence can be determined by using one of the sequence algorithms described herein (or other algorithms available to those of ordinary skill in the art) or by visual inspection. In a particular embodiment, a polypeptide sequence will have about 80%, about 85% or more, about 90% or more, about 95% or more, and about 98% or more, sequence identity to another polypeptide sequence.

As applied to polypeptides, the term substantial identity or substantial similarity means that two peptide sequences, when optimally aligned, such as by the programs BLAST, GAP or BESTFIT using default gap weights or by visual inspection, share sequence identity or sequence similarity. Similarly, as applied in the context of two nucleic acids, the term substantial identity or substantial similarity means that the two nucleic acid sequences, when optimally aligned, such as by the programs BLAST, GAP or BESTFIT using default gap weights (described elsewhere herein) or by visual inspection, share sequence identity or sequence similarity.

One example of an algorithm that is suitable for determining percent sequence identity or sequence similarity is the FASTA algorithm, which is described in Pearson, W. R. & Lipman, D. J., (1988) Proc. Natl. Acad. Sci. USA 85:2444. See also, W. R. Pearson, (1996) Methods Enzymology 266:227-258. Preferred parameters used in a FASTA alignment of DNA sequences to calculate percent identity or percent similarity are optimized, BL50 Matrix 15: −5, k-tuple=2; joining penalty=40, optimization=28; gap penalty −12, gap length penalty=−2; and width=16.

Another example of a useful algorithm is PILEUP. PILEUP creates a multiple sequence alignment from a group of related sequences using progressive, pairwise alignments to show relationship and percent sequence identity or percent sequence similarity. It also plots a tree or dendrogram showing the clustering relationships used to create the alignment. PILEUP uses a simplification of the progressive alignment method of Feng & Doolittle, (1987) J. Mol. Evol. 35:351-360. The method used is similar to the method described by Higgins & Sharp, CABIOS 5:151-153, 1989. The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. Using PILEUP, a reference sequence is compared to other test sequences to determine the percent sequence identity (or percent sequence similarity) relationship using the following parameters: default gap weight (3.00), default gap length weight (0.10), and weighted end gaps. PILEUP can be obtained from the GCG sequence analysis software package, e.g., version 7.0 (Devereaux et al., (1984) Nuc. Acids Res. 12:387-395).

Another example of an algorithm that is suitable for multiple DNA and amino acid sequence alignments is the CLUSTALW program (Thompson, J. D. et al., (1994) Nuc. Acids Res. 22:4673-4680). CLUSTALW performs multiple pairwise comparisons between groups of sequences and assembles them into a multiple alignment based on sequence identity. Gap open and Gap extension penalties were 10 and 0.05 respectively. For amino acid alignments, the BLOSUM algorithm can be used as a protein weight matrix (Henikoff and Henikoff, (1992) Proc. Natl. Acad. Sci. USA 89:10915-10919).

The term “foreign antigen” as used herein, refers to an antigen that originates from outside the body. Examples of foreign antigens include parts of or substances produced by viruses or microorganisms (such as bacteria and protozoa), as well as substances in snake venom, certain proteins in foods, and components of serum and red blood cells from other individuals.

The term “endogenous antigen” refers to an antigen that originate from the subject's own cells. Examples of endogenous antigens include, but are not limited to, cancer or tumor antigens, and antigens associated with an immune disease.

The term “effective amount” as used herein, refers to an amount that is sufficient to produce at least a reproducibly detectable amount of the desired result or effect. An effective amount will vary with the specific conditions and circumstances. Such an amount can be determined by the skilled practitioner for a given situation.

The terms “patient”, “subject” and “individual” are used interchangeably herein, and refer to an animal, particularly a human, to whom treatment including prophylaxis treatment (e.g., vaccination) is provided. This includes human and non-human animals. The term “non-human animals” as used herein includes all vertebrates, e.g., mammals, such as non-human primates, (particularly higher primates), sheep, dog, rodent (e.g., mouse or rat), guinea pig, goat, pig, cat, rabbits, cows, and non-mammals such as chickens, amphibians, reptiles etc. In one embodiment, the subject is human. In another embodiment, the subject is an experimental animal or animal substitute as a disease model. “Mammal” refers to any animal classified as a mammal, including humans, non-human primates, domestic and farm animals, and zoo, sports, or pet animals, such as dogs, cats, cattle, horses, sheep, pigs, goats, rabbits, etc. Patient or subject includes any subset of the foregoing, e.g., all of the above, but excluding one or more groups or species such as humans, primates or rodents. A subject can be male or female. A subject can be a fully developed subject (e.g., an adult) or a subject undergoing the developmental process (e.g., a child, infant or fetus).

The term “isolated” when used in reference to a nanoparticle-based vaccine disclosed herein, refers to the fact that the nanoparticle-based vaccine is separated from most other cellular components from which it was generated or in which it is typically present in nature. The nanoparticle-based vaccine disclosed herein are typically prepared to the state where they are substantially isolated to be completely isolated from most other cellular components and cellular debris.

The term “therapeutically effective amount” as used herein, refers to an amount that is sufficient to affect a therapeutically significant reduction in one or more symptoms of the condition when administered to a typical subject who has the condition. A therapeutically significant reduction in a symptom is, e.g., about 10%, about 20%, about 30%, about 40%, about 50%, about 60%, about 70%, about 80%, about 90%, about 100%, or more as compared to a control or non-treated subject.

The term “treat” or “treatment” as used herein, refers to a therapeutic treatment wherein the object is to eliminate or lessen symptoms. Beneficial or desired clinical results include, but are not limited to, elimination of symptoms, alleviation of symptoms, diminishment of extent of condition, stabilized (i.e., not worsening) state of condition, delay or slowing of progression of the condition.

Hydrogels with three-dimensional (3D) polymeric networks are formed via chemical or physical crosslinking and can hold a large amount of water or biological fluids without disintegration under physiological environments. Because of their high water content, excellent biocompatibility, and soft matter nature, hydrogels impart a biomimetic microenvironment similar to in vivo tissues, making them suitable for a variety of uses in the biomedical field, such as drug delivery, wound repair, tissue engineering, and 3D cell culture. Unlike surgical implantation of prefabricated hydrogels, which is invasive, inconvenient and painful for patients, injectable hydrogels that can be administered using a conventional syringe significantly improve patient compliance and comfort and minimize the risk of infection. Generally, in situ gelling systems exhibit the form of aqueous polymer solutions prior to injection and convert into non-flowing hydrogels upon exposure to environmental stimuli, such as temperature, pH, or by in situ chemical crosslinking based on photo-induced polymerization, Michael addition, Schiff base, enzyme, and so on. Among these hydrogels, thermosensitive hydrogels that exhibit reversible sol-gel transitions with increasing temperature are particularly attractive in view of their unique advantages, such as avoiding any chemical reaction and not involving the use of organic solvents. Therapeutic agents, bioactive molecules, or cells can be entrapped via simply blending them with aqueous polymer solutions at low or room temperature and after being injected into the body, mixed solutions spontaneously turn into sustained drug delivery devices. The design of thermosensitive hydrogels for various biomedical applications often needs to satisfy some important requirements: (a) viscosity of aqueous polymer solutions should be sufficiently low to facilitate a homogeneous dispersion with drugs/cells and the subsequent injection; (b) fast gelation upon exposure to body temperature is required to minimize the initial burst effect of loaded drugs and the leakage of encapsulated cells; (c) they should be biocompatible and can be degraded after achieving their intended purposes, and their degradation products should be non-cytotoxic; and (d) they should provide controlled drug release profiles to match different biomedical applications.

Although vaccines are effective to protect against infectious diseases, they can also be limited in their efficacy over time, requiring multiple boosters or new immunizations as variants drift. While protein subunit vaccines are often considered the safest design approach (vs. attenuated/inactive microorganisms), they can struggle to induce robust immune responses to immunization. This can be overcome with the use of adjuvants (e.g., MPLA, MF59, or CpG), or delivery vehicles, such as protein-based NPs. This class of particles have virus-like nanostructures but lack infectious genetic materials, providing safety while inducing humoral or cellular immune responses through the recognition of repetitive subunits on the particle surface. NPs with diameters ranging from ˜20- to 50-nm are optimally sized to drain into lymph nodes and be taken up by dendritic cells (DCs), making them ideal scaffolds for delivering antigens and immunostimulants.

The disclosure provides for a vaccine depot formulation or preparation that promotes effective immunization in a subject by providing for the sustained release of an antigen and adjuvant from nanoparticles embedded in a biodegradable thermosensitive hydrogel. Further, by incorporating the antigen and adjuvant into a nanoparticle, a synergy can be achieved for the simultaneous delivery of the antigen and adjuvant, together with a hydrogel depot for modulating the nanoparticle release kinetics. The sustained release will change the kinetics of the antigen and adjuvant exposure to the immune system, resulting in significantly longer lasting (more durable) and broader cross-reactive immune responses, both of which increase the efficacy of vaccines.

In a particular embodiment the disclosure provides a vaccine depot formulation or preparation that provides for the simultaneous delivery of an antigen and an adjuvant in vivo, comprising: a biodegradable thermosensitive hydrogel that has been loaded or embedded with nanoparticles that comprise the antigen and the adjuvant, wherein the nanoparticles are hollow and comprise an outer surface and an accessible inner cavity, where the antigen and/or the adjuvant is conjugated to the outer surface of the nanoparticles, and/or where the antigen and/or the adjuvant is conjugated or loaded into the inner cavity of the nanoparticles, wherein the antigen is designed to promote a targeted immune response when the vaccine depot is administered in vivo. In a particular embodiment, each nanoparticle comprises 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, or 60, or a range that includes or is between any two of the foregoing values (e.g., from 15 to 30), of conjugated adjuvant molecules or compounds (e.g., CpG compounds). In further embodiments, each nanoparticle may comprise 2, 3, 4, 5, 6, 7, 8, 9, 10, or a range that includes or is between any two of the foregoing values (e.g., from 2 to 5), different type of conjugated antigens (e.g., different CpG compounds, different classes of adjuvants, etc.). In another embodiment, each nanoparticle comprises 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, or 60, or a range that includes or is between any two of the foregoing values (e.g., from 15 to 30), of conjugated antigen molecules or compounds (e.g., H5, CBU1910, etc.). In additional embodiments, each nanoparticle may comprise 2, 3, 4, 5, 6, 7, 8, 9, 10, or a range that includes or is between any two of the foregoing values (e.g., from 2 to 4), different type of conjugated antigens (e.g., diptheria antigens, tetanus antigens, and pertussis antigens; measles antigens, mumps antigens, and rubella antigens, etc.). In further embodiment, nanoparticles comprising conjugated antigen and adjuvant have a hydrodynamic size of about 30 nm, 31 nm, 32 nm, 33 nm, 34 nm, 35 nm, 36 nm, 37 nm, 38 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, or 100 nm, or range that includes or is between any two of the foregoing sizes, including fractional increments thereof. In a particular embodiment, the nanoparticles comprising conjugated antigen and adjuvant have a hydrodynamic size from 35 nm to 60 nm.

In a further embodiment, the vaccine depot formulation of the disclosure is suitable for injection by the biodegradable thermosensitive hydrogel being in a liquid state at normal ambient temperatures but solidifying into a gel-like state at a normal body temperature. In yet a further embodiment, the biodegradable thermosensitive hydrogel is in a liquid state at a temperature less than 30° C. Examples of biodegradable thermosensitive hydrogels include, but are not limited to, poloxamer-407/188, poloxamer-407, chitosan, poloxamer-chitosan, cellulose, methylcellulose, sodium carboxymethyl cellulose, PLGA-PEG-PLGA (PPP), PCL-PEG-PCL, and poly(N-isopropylacrylamide). In a particular embodiment, the biodegradable thermosensitive hydrogel is PPP. PPP-based polymers have been investigated to extend in vivo release for small molecule drugs, peptides, and small protein therapeutics, but the use of PPP-based polymers for the controlled-release of antigen and adjuvant in vaccine-based applications has not been reported. Previous studies using uncoupled antigen and adjuvant (only mixed in polymer) could yield differential release kinetics, leading to suboptimal spatial and temporal delivery. In direct contrast, the vaccine depot formulation or preparation disclosed herein allows for simultaneous co-delivery of antigen and adjuvant to antigen-presenting cells (APCs), yielding higher APC activation.

In the studies presented herein, it was found that use of a biodegradable thermosensitive hydrogel to embed nanoparticles comprising an adjuvant and an antigen provided significantly better results in comparison to nanoparticles comprising an adjuvant and an antigen but not embedded in a biodegradable thermosensitive hydrogel. For example, an antigen and an adjuvant (IVAX-1) embedded in a PPP hydrogel elicited higher total IgG, IgG1 and IgG2c than a control group without PPP gel. The hydrogel depot formulation also elicited both broader homosubtypic and heterosubtypic cross-reactivity to HA (H1, H2 and H7). In further studies presented herein, a Coxiella burnetii antigen (CBU1910) antigen was conjugated to the interior of a E2 nanoparticle via CpG, which was then embedded in a PPP hydrogel. This CBU1910-CpG-E2 hydrogel depot formulation exhibited stronger antigen specific IgG, IgG1, and IgG2c responses after immunization than free antigen, free antigen embedded in a hydrogel, and the CBU1910-CpG-E2 nanoparticles that were not embedded in a hydrogel. Up to week 16 after vaccination, antigen specific IgG is still higher in animals vaccinated with the CBU1910-CpG-E2 hydrogel depot formulation than the control groups. Additional studies with a H5-CpG-E2 hydrogel depot formulation demonstrated in comparison to H5-CpG-E2 nanoparticles alone, showed an increase in total IgG and a more balanced IgG1/IgG2c ratio. Additionally, mice treated with a H5-CpG-E2 hydrogel depot formulation had a much higher survival rate and had lower weight loss than mice treated with H5-CpG-E2 nanoparticles. The results demonstrate that a vaccine depot formulation or preparation of disclosure when targeted antigen(s) are embedded in a hydrogel provides longer lasting protection than soluble nanoparticle-based vaccines. The results further demonstrate that a vaccine depot formulation or preparation of disclosure when targeted antigen(s) are incorporated into nanoparticles which are embedded in a hydrogel provides even longer lasting protection than all other types of tested formulations.

Moreover, the vaccine depot formulation or preparation has higher antigen loading amounts compared with other platforms, e.g., microneedle array, have high efficiencies, and are cheaper and faster to manufacture and easier to use compared with other platforms, e.g., osmosis pump and other implanted devices. Furthermore, the thermosensitive hydrogels used the vaccine depot formulation or preparation of the disclosure are characterized by having good safety profiles in vivo. For example, PLGA and PEG components of the hydrogels used in the studies presented are FDA-approved. Moreover, it is expected that choice of the thermosensitive hydrogels or the hydrogel components thereof, can be used to realize the most favorable kinetic release rates of the targeted antigen(s).

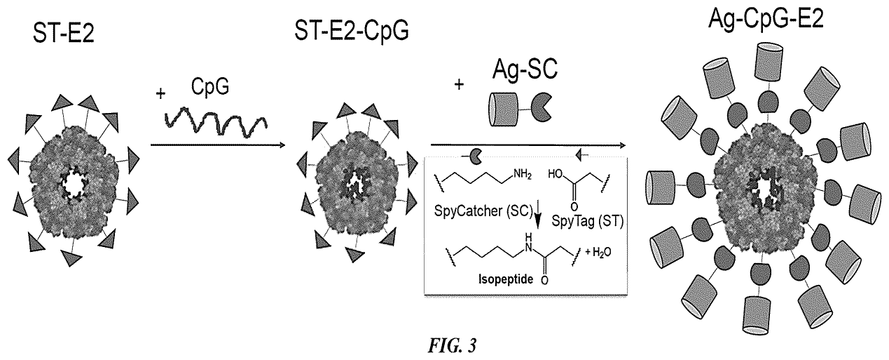

It should be understood, that while influenza H5 and Coxiella antigens were used in the Examples presented herein, any antigen can be conjugated to the nanoparticle of the disclosure by using the same or similar protocols disclosed herein. For example, a SpyCatcher peptide can be attached to the antigen to form an Ag-SC that can then conjugate to SpyTag of the nanoparticles disclosed herein in the same strategy presented in FIG. 3. Antigens that can be used with a vaccine depot disclosed herein are typically foreign or exogenous antigens from a pathogen, or endogenous antigens from a noninfectious disease (e.g., cancer or immune disease antigens). In a further embodiment, the foreign or exogenous antigens are from a pathogen selected from a fungus, virus, or bacterium. In yet a further embodiment, the pathogen is a bacterium selected from the following: Actinomyces israelii, Bacillus anthracis, Bacillus cereus, Bartonella henselae, Bartonella quintana, Bordetella pertussis, Borrelia burgdorferi, Borrelia garinii, Borrelia afzelii, Borrelia recurrentis, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diphtheriae, Enterococcus faecalis, Enterococcus faecium, Escherichia coli, Francisella tularensis, Haemophilus influenzae, Helicobacter pylori, Legionella pneumophila, Leptospira interrogans, Leptospira santarosai, Leptospira weilii, Leptospira noguchii, Listeria monocytogenes, Mycobacterium leprae, Mycobacterium tuberculosis, Mycobacterium ulcerans, Mycoplasma pneumoniae, Neisseria gonorrhoeae, Neisseria meningitidis, Pseudomonas aeruginosa, Rickettsia rickettsia, Salmonella typhi, Salmonella typhimurium, Shigella sonnei, Staphylococcus aureus, Staphylococcus epidermidis, Staphylococcus saprophyticus, Streptococcus agalactiae, Streptococcus pneumoniae, Streptococcus pyogenes, Treponema pallidum, Ureaplasma urealyticum, Vibrio cholerae, Yersinia pestis, Yersinia enterocolitica, and Yersinia pseudotuberculosis. In another embodiment, the pathogen is a fungus selected from the following: Absidia corymbifera, Absidia ramose, Achorion gallinae, Actinomadura spp., Ajellomyces dermatididis, Aleurisma brasiliensis, Allersheria boydii, Arthroderma spp., Aspergillus flavus, Aspergillus fumigatu, Basidiobolus spp, Blastomyces spp, Cadophora spp, Candida albicans, Cercospora apii, Chrysosporium spp, Cladosporium spp, Cladothrix asteroids, Coccidioides immitis, Cryptococcus albidus, Cryptococcus gattii, Cryptococcus laurentii, Cryptococcus neoformans, Cunninghamella elegans, Dematium wernecke, Discomyces israelii, Emmonsia spp, Emmonsiella capsulate, Endomyces geotrichum, Entomophthora coronate, Epidermophyton floccosum, Filobasidiella neoformans, Fonsecaea spp., Geotrichum candidum, Glenospora khartoumensis, Gymnoascus gypseus, Haplosporangium parvum, Histoplasma, Histoplasma capsulatum, Hormiscium dermatididis, Hormodendrum spp., Keratinomyces spp, Langeronia soudanense, Leptosphaeria senegalensis, Lichtheimia corymbifera, Lobmyces loboi, Loboa loboi, Lobomycosis, Madurella spp., Malassezia furfur, Micrococcus pelletieri, Microsporum spp, Monilia spp., Mucor spp., Mycobacterium tuberculosis, Nannizzia spp., Neotestudina rosatii, Nocardia spp., Oidium albicans, Oospora lactis, Paracoccidioides brasiliensis, Petriellidium boydii, Phialophora spp., Piedraia hortae, Pityrosporum furfur, Pneumocystis jirovecii (or Pneumocystis carinii), Pullularia gougerotii, Pyrenochaeta romeroi, Rhinosporidium seeberi, Sabouraudites (Microsporum), Sartorya fumigate, Sepedonium, Sporotrichum spp., Stachybotrys, Stachybotrys chartarum, Streptomyce spp., Tinea spp., Torula spp, Trichophyton spp, Trichosporon spp, and Zopfia rosatii. In yet another embodiment, the pathogen is a virus selected from the following: Human coronavirus, Human papillomavirus, Torque teno virus, Barmah forest virus, Chikungunya virus, Eastern equine encephalitis virus, Mayaro virus, O'nyong-nyong virus, Ross river virus, Sagiyama virus, Semliki forest virus, Sindbis virus, Venezuelan equine encephalitis virus, Western equine encephalitis virus, Junin arenavirus, Lassa virus, Lymphocytic choriomeningitis virus, Machupo virus, Pichinde virus, Human SARS coronavirus, MERS coronavirus, SARS coronavirus, Encephalomyocarditis virus, Cosavirus A, Human cytomegalovirus, Human T-lymphotropic virus, Hepatitis delta virus, Adeno-associated virus, Ebolavirus, Human rhinovirus, Coxsackievirus, Echovirus, Human enterovirus, Poliovirus, Human parvovirus B19, Murray valley encephalitis virus, Dengue virus, Japanese encephalitis virus, Langat virus, Louping ill virus, St. louis encephalitis virus, Tick-borne powassan virus, West Nile virus, Yellow fever virus, Zika virus, Hantaan virus, New York virus, Puumala virus, Seoul virus, Hendra virus, Nipah virus, Hepatitis virus, Influenza virus, Avian influenza A, Aichi virus, Human immunodeficiency virus, Cercopithecine herpesvirus, Epstein-Barr virus, Australian bat lyssavirus, Duvenhage virus, Lagos bat virus, Mokola virus, Rabies virus, European bat lyssavirus, Human astrovirus, Lake Victoria marburgvirus, Human adenovirus, Molluscum contagiosum virus, Measles virus, Human papillomavirus, Crimean-Congo hemorrhagic fever virus, Dugbe virus, Norwalk virus, Southampton virus, Oropouche virus, Bunyamwera virus, Bunyavirus La Crosse, Bunyavirus snowshoe hare, Hepatitis B virus, Human respiratory syncytial virus, Monkeypox virus, Cowpox virus, Horsepox virus, Vaccinia virus, Variola virus, Yaba monkey tumor virus, Yaba-like disease virus, Orf virus, GB virus C/Hepatitis G virus, Punta toro phlebovirus, Rift valley fever virus, Sandfly fever Naples phlebovirus (Toscana virus), Sandfly fever sicilian virus, Uukuniemi virus, BK polyomavirus, JC polyomavirus, KI Polyomavirus, Merkel cell polyomavirus, WU polyomavirus, Human parainfluenza, Rosavirus, Human herpesvirus, Rotavirus, Rubella virus, Mammalian orthorubulavirus (Simian virus), Mumps virus, Salivirus A, Sapporo virus, Banna virus, Eastern chimpanzee simian foamy virus, Simian foamy virus, Dhori virus, Vientovirus, Human torovirus, Varicella-zoster virus, Chandipura virus, Isfahan virus, and Vesicular stomatitis virus. In a particular embodiment, the foreign antigens are from an influenza virus (e.g., hemagglutinin from influenza), from a human SARS coronavirus (e.g., spike protein from SARS-CoV-2), or from Coxiella burnetii (e.g., CBU1910).

In a further embodiment, the vaccine depot formulation or preparation comprises an adjuvant along with the antigen conjugated to the nanoparticles, wherein the adjuvant increases or modulates the immune response in subjects receiving the vaccine. Adjuvants in immunology are often used to modify or augment the effects of a vaccine by stimulating the immune system to respond to the vaccine more vigorously, and thus providing increased immunity to a particular disease. Adjuvants accomplish this task by mimicking specific sets of evolutionarily conserved molecules, so called pathogen-associated molecular patterns, which include liposomes, lipopolysaccharide, molecular cages for antigens, components of bacterial cell walls, and endocytosed nucleic acids such as RNA, double-stranded RNA, single-stranded DNA, and unmethylated CpG dinucleotide-containing DNA. Because immune systems have evolved to recognize these specific antigenic moieties, the presence of an adjuvant in conjunction with the vaccine can greatly increase the innate immune response to the antigen by augmenting the activities of dendritic cells, lymphocytes, and macrophages by mimicking a natural infection. Examples of adjuvants include, but are not limited to, CpG oligodeoxynucleotide based adjuvants, ssRNA, aluminum salts (e.g., aluminum hydroxide, aluminum phosphate, and aluminum potassium sulfate), AS04, MF59, AS01_{B}, and CpG 1018. In a particular embodiment, the adjuvant is loaded or conjugated to the inner cavity and/or the outer surface of the nanoparticles by use of a linker that binds to both the adjuvant and free cysteine groups on the outer surface and/or inner cavity of the nanoparticles. In yet another embodiment, the adjuvant has been modified to comprise a terminal aldehyde or benzaldehyde group, and wherein the linker is a N-(β-maleimidopropionic acid) hydrazide (BMPH) linker.

In another embodiment, the vaccine depot formulation or preparation of the disclosure provides for the simultaneous delivery of the antigen and adjuvant from the nanoparticle in vivo. It was found in the studies presented herein that the simultaneous delivery of the antigen and adjuvant from the nanoparticle using the vaccine depot formulation of the disclosure provided for unexpected results in that the immune response was greatly improved when both agents were conjugated to the nanoparticles as opposed to only one agent being conjugated to the nanoparticles and the other agent being administered separately, even if the other agent was administered at the same time as the nanoparticle.

In a particular embodiment, the vaccine depot formulation or preparation disclosed herein comprises an antigen and a CpG oligodeoxynucleotide based adjuvant that are conjugated to nanoparticles. Examples of CpG oligodeoxynucleotide based adjuvants include, but are not limited to, CpG1018, CpG1826, CpG1018, CpG1826, CpG ODN 1466, CpG ODN PB3, CpG ODN BW005, CpG Alum, CPG 21424, CpG 7909, CpG-ODN 2135, ODN K3, CpG ODN 10101, CpG-28, CpG ODN C274, CpG ODN C695, CpG ODN #17, CpG-ODN 2722, CpG 8916, CpG 8954, CpG ODN 678, CpG ODN BW015, CpG ODN 658, CpG ODN 640, CpG ODN PB9, CpG ODN BW004, CpG ODN BW103, CpG ODN 110, CpG ODN BW206, CpG ODN 607, CpG ODN 647, CpG ODN 111, CpG ODN 109, CpG ODN 656, CpG ODN 664, CpG ODN C9, CPG2429, CPG5475, CPG21608, CPG21797, CPG21796, CPG21799, CPG21800, CPG21802, CPG21889, CPG23409, CpG-c41, CPG23410, CPG23411, CPG23412, CPG23413, CPG23617, CPG23414, CPG 1681, CPG 2143, CPG 21425, and CPG 21426. The sequences and activities for the foregoing CpG oligodeoxynucleotide based adjuvants are known and can readily be found in the art (e.g., see BOC Sciences). In a further embodiment, the CpG oligodeoxynucleotide based adjuvant has the sequence of SEQ ID NO:40 and comprises a terminal 5′ benzaldehyde.

Synthetic oligodeoxynucleotides (ODNs) containing unmethylated CpG motifs trigger cells that express Toll-like receptor 9 (including human plasmacytoid dendritic cells and B cells) to mount an innate immune response characterized by the production of Th1 and proinflammatory cytokines. When used as vaccine adjuvants, CpG ODNs improve the function of professional antigen-presenting cells and boost the generation of humoral and cellular vaccine-specific immune responses. These effects are optimized by maintaining ODNs and vaccine in close proximity. CpG DNA directly activates pDCs and B cells, contributing to the induction of both innate and adaptive immune responses. The cascade of events initiated by CpG DNA indirectly supports the maturation, differentiation and proliferation of natural killer cells, T cells and monocytes/macrophages. TLR-9-stimulated B cells produce IL-6, IL-12 and the CXCR3 chemokines IP-10, Mig and I-TAC. They also secrete IgM in a process partially dependent on IL-6 expression. B cells activated by CpG DNA upregulate expression of their Fc receptor (FcR) and costimulatory molecules including MHC class II, CD40, CD80 and CD86. Subsequently, the CpG-stimulated B cells proliferate and differentiate into plasma cells and memory B cells. CpG-based adjuvants can also impact cellular immune responses. In a recent study, the number and survival of CD8⁺ T cells was significantly enhanced when CpG ODN was administered 2 days before peptide vaccination. The persistence of the CD8⁺ T cells in the circulation doubled when compared with vaccine alone (p<0.001). These effects were not observed with other TLR ligands.

In a particular embodiment, the vaccine depot formulation or preparation disclosed herein comprises nanoparticles that are hollow and comprise an outer surface and an accessible inner cavity, where the antigen and/or the adjuvant is conjugated to the outer surface of the nanoparticles, and/or where the antigen and/or the adjuvant is conjugated or loaded into the inner cavity of the nanoparticles. In a further embodiment, the antigen is conjugated to the outer surface of the nanoparticles, and wherein the adjuvant is conjugated or loaded into the inner cavity of the nanoparticles. In yet a further embodiment, the adjuvant is conjugated to the outer surface of the nanoparticles, and wherein the antigen is conjugated or loaded into the inner cavity of the nanoparticles. In another embodiment, the nanoparticles are hollow protein nanoparticles. In yet a further embodiment, the hollow protein nanoparticles are based on ferritin, TIP60, or hepatitis B virus (HBV) surface-antigen protein. In another embodiment, the hollow protein nanoparticles are based on the E2 subunit of the pyruvate dehydrogenase complex (PDC) from the Geobacillus stearothermophilus. The E2 subunit from Geobacillus stearothermophilus can self-assemble into a 60-mer hollow spherical protein cage of ˜25 nm diameter and can be functionalized with non-native molecules on its external and internal surfaces. This platform has been shown to efficiently activate dendritic cells and elicit CD8 T cell responses in tumor vaccination models when using CD8 epitope peptide antigens. In a further embodiment, the lipoyl domain of the E2 subunit has been removed resulting in a E2 protein that comprises 256 amino acids having the sequence of SEQ ID NO:44. In a further embodiment, the amino acids MLSV have been added 5′ end of the sequence presented in SEQ ID NO:44 to provide the sequence of SEQ ID NO:45. In order to facilitate the conjugation of the adjuvant and antigen to the E2 nanoparticles, one or more amino acids of the E2 subunit have been recombinantly modified to substitute one or more amino acids with cysteines. In a further embodiment, the recombinant E2 subunit has a polypeptide sequence that is at least 95%, at least 98%, or at least 99% identical to the sequence presented in SEQ ID NO:5 and which has an amino acid substitution of D214C in comparison to SEQ ID NO:45. In a certain embodiment, the recombinant E2 subunit has a polypeptide sequence of SEQ ID NO:5, SEQ ID NO:7, or SEQ ID NO:9. In a further embodiment, the recombinant E2 subunit has a polypeptide sequence of SEQ ID NO:5.

In a certain embodiment, the agent(s) (e.g., antigen and/or adjuvant) can be conjugated to the nanoparticles by using any number of methods and reagents known in the art. For example, the methods and reagents for conjugating the agent(s) (e.g., antigen and/or adjuvant) to the nanoparticles can include, but are not limited to, mal-tNTA-Ni, sulfo-SMCC, sortase A ligation, and the SpyCatcher/SpyTag system. The SpyCatcher-SpyTag system was developed seven years ago as a method for protein ligation. It is based on a modified domain from a Streptococcus pyogenes surface protein (SpyCatcher), which recognizes a cognate 13-amino-acid peptide (SpyTag). Upon recognition, the two forma a covalent isopeptide bond between the side chains of a lysine in SpyCatcher and an aspartate in SpyTag. The SpyTag system is versatile as the tag is a short, unfolded peptide that can be genetically fused to exposed positions in target proteins; similarly, SpyCatcher can be fused to an antigen disclosed herein. The SpyTag/SpyCatcher system was used to attach an antigen, such as a foreign or endogenous antigen, to the E2 nanoparticle using the strategy outlined in FIG. 3. The advantages of this approach include its stable covalent interaction and the ability to separately express both the antigen and the nanoparticle proteins prior to conjugation, which can circumvent protein expression challenges. In the examples presented herein, SpyTag (ST) was genetically attached to the E2 nanoparticle, and SpyCatcher (SC) was genetically fused with an antigen (e.g., CBU1910 or H5). Alternatively, the SpyCatcher (SC) can be genetically attached to the E2 nanoparticle, and SpyTag (ST) can be genetically fused with an antigen. It was postulated that coupling SpyCatcher to the antigen would minimize the amount of exposed SC after conjugation to the NP, which is likely favorable for reducing anti-SC immune responses. It should be understood, that while influenza H5 and Coxiella antigens were used in the Examples presented herein, any antigen can be conjugated to the nanoparticle of the disclosure by using the same or similar protocols disclosed herein. For example, a SpyCatcher peptide can be attached to any desired antigen to form an Ag-SC that can then conjugate to SpyTag of the nanoparticles disclosed herein using the same strategy presented in FIG. 3. In a particular embodiment, the outer surface of the nanoparticles is conjugated with a SpyTag peptide, and wherein the antigen is conjugated with a SpyCatcher peptide. In a further embodiment, the SpyTag peptide has the sequence of SEQ ID NO:17, and wherein the SpyCatcher peptide has the sequence of SEQ ID NO: 19. In another embodiment, the SpyTag conjugated recombinant E2 protein nanoparticles (ST-E2) have the sequence of SEQ ID NO:21. In yet another embodiment, the antigen that is conjugated with a SpyCatcher peptide is CBU1910 and the construct (SC-CBU1910) has the sequence of SEQ ID NO:23. In a further embodiment, the antigen that is conjugated with a SpyCatcher peptide is H5 hemagglutinin and the construct (SC-H5) has the sequence of SEQ ID NO:43.

In a particular embodiment, the disclosure also provides a method of immunizing a subject comprising: administering one or more doses of the vaccine depot formulation of the disclosure to protect a subject from an infection by a pathogen and/or from a disease. In a further embodiment, the infection and/or disease is selected from Q fever, avian influenza, and SARS-CoV-2. In yet a further embodiment, the vaccine depot formulation is formulated for intramuscular administration, subcutaneous administration, intravenous administration, or other forms of administration. In another embodiment, the vaccine depot formulation provides long-lasting, sustained delivery of the antigen for at least 2, 4, 6, 8, 10, or 12 weeks in vivo.

The disclosure further provides for specified modes of administration for administering the vaccine depot formulation or preparation disclosed herein. In one embodiment, the disclosure provides for a pharmaceutical composition that comprises a vaccine depot formulation or preparation disclosed herein and a pharmaceutically acceptable carrier. The term “pharmaceutically acceptable carrier” as used herein means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the composition and is compatible with administration to a subject, for example a human. Such compositions can be specifically formulated for administration via one or more of a number of routes, such as the routes of administration described herein. Supplementary active ingredients also can be incorporated into the compositions. When an agent, formulation or pharmaceutical composition described herein, is administered to a subject, preferably, a therapeutically effective amount is administered. As used herein, the term “therapeutically effective amount” refers to an amount that result in an improvement or remediation of the condition.

The disclosure further provides for the use of a vaccine depot formulation or preparation disclosed herein for vaccinating a subject. Suitable methods of administering a vaccine depot formulation or preparation described herein to a patient include by any route of in vivo administration that is suitable for delivering thermosensitive hydrogels to a patient. Examples of modes of administration include, but are not limited to, intravenous administration, intertumoral administration, intraperitoneal administration, intramuscular administration, intracoronary administration, intraarterial administration (e.g., into a carotid artery), subcutaneous administration, transdermal delivery, intratracheal administration, subcutaneous administration, intraarticular administration, intraventricular administration, inhalation (e.g., aerosol), nasal, oral, pulmonary administration, impregnation of a catheter, and direct injection into a tissue.

Intravenous, intraperitoneal, and intramuscular administrations can be performed using methods standard in the art. Aerosol (inhalation) delivery can also be performed using methods standard in the art (see, for example, Stribling et al., Proc. Natl. Acad. Sci. USA 189: 11277-11281, 1992, which is incorporated herein by reference in its entirety). Oral delivery can be performed by complexing a vaccine depot formulation or preparation disclosed herein to a carrier capable of withstanding degradation by digestive enzymes in the gut of an animal. Examples of such carriers include plastic capsules or tablets, such as those known in the art.

The appropriate dosage and treatment regimen for the vaccine depot formulation or preparation described herein will vary with respect to the needed vaccination schedule of the subject. In certain cases, only one vaccine depot formulation or preparation may need to be administered to a subject to bring about effective immunity to a pathogen or noninfectious disease. In other cases, one or more booster shots of the vaccine depot formulation or preparation disclosed herein may be needed. In such a case, the one or more booster shots may have the same dose of targeted antigen(s) or be of a lower dose. For multiple doses of the vaccine depot formulation or preparation disclosed herein, they may be administered a week or more apart.

For use in the therapeutic or biological applications described herein, kits and articles of manufacture are also described herein. Such kits can comprise a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. The containers can be formed from a variety of materials such as glass or plastic.

For example, the container(s) can comprise a vaccine depot formulation or preparation system described herein, optionally in a composition or in combination with another agent as disclosed herein. The container(s) optionally have a sterile access port (for example the container can be an intravenous solution bag or a vial having a stopper pierceable by a hypodermic injection needle). Such kits optionally comprise an identifying description or label or instructions relating to its use in the methods described herein.

A kit will typically comprise one or more additional containers, each with one or more of various materials (such as reagents, optionally in concentrated form, and/or devices) desirable from a commercial and user standpoint for use of a compound described herein. Non-limiting examples of such materials include, but are not limited to, buffers, diluents, filters, needles, syringes; carrier, package, container, vial and/or tube labels listing contents and/or instructions for use, and package inserts with instructions for use. A set of instructions will also typically be included.

A label can be on or associated with the container. A label can be on a container when letters, numbers or other characters forming the label are attached, molded or etched into the container itself, a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein. These other therapeutic agents may be used, for example, in the amounts indicated in the Physicians' Desk Reference (PDR) or as otherwise determined by one of ordinary skill in the art.

In a certain embodiment, the disclosure also provides a method to make the vaccine depot formulations of the disclosure, comprising: reacting SpyTag conjugated recombinant E2 protein (ST-E2) nanoparticles comprising a plurality of free cysteine groups with a linker and adjuvant comprising a terminal aldehyde or benzaldehyde group, to form ST-E2 nanoparticles comprising the adjuvant; and reacting the ST-E2 nanoparticles comprising the adjuvant with a SpyCatcher conjugated antigen to form the vaccine depot formulation. In a further embodiment, the ST-E2 nanoparticles have the sequence of SEQ ID NO:21. In yet a further embodiment, the linker is a N-(β-maleimidopropionic acid) hydrazide (BMPH) linker. In another embodiment, the adjuvant has the sequence of SEQ ID NO:40 that comprises a terminal 5′ benzaldehyde. In yet a further embodiment, the SpyCatcher conjugated antigen comprises a foreign antigen from a pathogen or an endogenous antigen from a cancer or is associated with an immune disease. In another embodiment, a SpyCatcher peptide that has the sequence of SEQ ID NO:19 is conjugated to the antigen.

In another embodiment, the disclosure also provides a method to make the vaccine depot formulations of the disclosure, comprising: reacting SpyTag conjugated recombinant E2 protein (ST-E2) nanoparticles comprising the adjuvant with a SpyCatcher conjugated antigen to form the vaccine depot formulation. In a further embodiment, the ST-E2 nanoparticles have the sequence of SEQ ID NO:21. In yet a further embodiment, the linker is a N-(β-maleimidopropionic acid) hydrazide (BMPH) linker. In another embodiment, the adjuvant has the sequence of SEQ ID NO:40 that comprises a terminal 5′ benzaldehyde. In yet a further embodiment, the SpyCatcher conjugated antigen comprises a foreign antigen from a pathogen or an endogenous antigen from a cancer or is associated with an immune disease. In another embodiment, a SpyCatcher peptide that has the sequence of SEQ ID NO:19 is conjugated to the antigen.

The disclosure further provides that the methods and compositions described herein can be further defined by the following aspects (aspects 1 to 47):

1. A vaccine depot formulation for the simultaneous delivery of an antigen and an adjuvant, comprising:
a biodegradable thermosensitive hydrogel that has been loaded or embedded with nanoparticles that comprise the antigen and the adjuvant,
wherein the nanoparticles are hollow and comprise an outer surface and an accessible inner cavity, where the antigen and/or the adjuvant is conjugated to the outer surface of the nanoparticles, and/or where the antigen and/or the adjuvant is conjugated or loaded into the inner cavity of the nanoparticles.

2. The vaccine depot formulation of aspect 1, wherein the vaccine depot formulation is suitable for injection by the biodegradable thermosensitive hydrogel being in a liquid state at normal ambient temperatures but solidifying into a gel-like state at a normal body temperature.

3. The vaccine depot formulation of aspect 2, wherein the biodegradable thermosensitive hydrogel is in a liquid state at a temperature less than 30° C.

4. The vaccine depot formulation of any one of aspects 1 to 3, wherein the biodegradable thermosensitive hydrogel is selected from poloxamer-407/188, poloxamer-407, chitosan, poloxamer-chitosan, cellulose, methylcellulose, sodium carboxymethyl cellulose, PLGA-PEG-PLGA (PPP), PCL-PEG-PCL, and poly(N-isopropylacrylamide).

5. The vaccine depot formulation of any one of aspects 1 to 4, wherein the biodegradable thermosensitive hydrogel is PPP.

6. The vaccine depot formulation of any one of aspects 1 to 5, wherein the antigen is a foreign antigen from a pathogen selected from a bacterium, a fungus or a virus.

7. The vaccine depot formulation of aspect 6, wherein the bacterium is selected from Actinomyces israelii, Bacillus anthracis, Bacillus cereus, Bartonella henselae, Bartonella quintana, Bordetella pertussis, Borrelia burgdorferi, Borrelia garinii, Borrelia afzelii, Borrelia recurrentis, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diphtheriae, Enterococcus faecalis, Enterococcus faecium, Escherichia coli, Francisella tularensis, Haemophilus influenzae, Helicobacter pylori, Legionella pneumophila, Leptospira interrogans, Leptospira santarosai, Leptospira weilii, Leptospira noguchii, Listeria monocytogenes, Mycobacterium leprae, Mycobacterium tuberculosis, Mycobacterium ulcerans, Mycoplasma pneumoniae, Neisseria gonorrhoeae, Neisseria meningitidis, Pseudomonas aeruginosa, Rickettsia rickettsia, Salmonella typhi, Salmonella typhimurium, Shigella sonnei, Staphylococcus aureus, Staphylococcus epidermidis, Staphylococcus saprophyticus, Streptococcus agalactiae, Streptococcus pneumoniae, Streptococcus pyogenes, Treponema pallidum, Ureaplasma urealyticum, Vibrio cholerae, Yersinia pestis, Yersinia enterocolitica, and Yersinia pseudotuberculosis.

8. The vaccine depot formulation of aspect 6, wherein the fungus is selected from Absidia corymbifera, Absidia ramose, Achorion gallinae, Actinomadura spp., Ajellomyces dermatididis, Aleurisma brasiliensis, Allersheria boydii, Arthroderma spp., Aspergillus flavus, Aspergillus fumigatu, Basidiobolus spp, Blastomyces spp, Cadophora spp, Candida albicans, Cercospora apii, Chrysosporium spp, Cladosporium spp, Cladothrix asteroids, Coccidioides immitis, Cryptococcus albidus, Cryptococcus gattii, Cryptococcus laurentii, Cryptococcus neoformans, Cunninghamella elegans, Dematium wernecke, Discomyces israelii, Emmonsia spp, Emmonsiella capsulate, Endomyces geotrichum, Entomophthora coronate, Epidermophyton floccosum, Filobasidiella neoformans, Fonsecaea spp., Geotrichum candidum, Glenospora khartoumensis, Gymnoascus gypseus, Haplosporangium parvum, Histoplasma, Histoplasma capsulatum, Hormiscium dermatididis, Hormodendrum spp., Keratinomyces spp, Langeronia soudanense, Leptosphaeria senegalensis, Lichtheimia corymbifera, Lobmyces loboi, Loboa loboi, Lobomycosis, Madurella spp., Malassezia furfur, Micrococcus pelletieri, Microsporum spp, Monilia spp., Mucor spp., Mycobacterium tuberculosis, Nannizzia spp., Neotestudina rosatii, Nocardia spp., Oidium albicans, Oospora lactis, Paracoccidioides brasiliensis, Petriellidium boydii, Phialophora spp., Piedraia hortae, Pityrosporum furfur, Pneumocystis jirovecii (or Pneumocystis carinii), Pullularia gougerotii, Pyrenochaeta romeroi, Rhinosporidium seeberi, Sabouraudites (Microsporum), Sartorya fumigate, Sepedonium, Sporotrichum spp., Stachybotrys, Stachybotrys chartarum, Streptomyce spp., Tinea spp., Torula spp, Trichophyton spp, Trichosporon spp, and Zopfia rosatii.

9. The vaccine depot formulation of aspect 6, wherein the virus is selected from Human coronavirus, Human papillomavirus, Torque teno virus, Barmah forest virus, Chikungunya virus, Eastern equine encephalitis virus, Mayaro virus, O'nyong-nyong virus, Ross river virus, Sagiyama virus, Semliki forest virus, Sindbis virus, Venezuelan equine encephalitis virus, Western equine encephalitis virus, Junin arenavirus, Lassa virus, Lymphocytic choriomeningitis virus, Machupo virus, Pichinde virus, Human SARS coronavirus, MERS coronavirus, SARS coronavirus, Encephalomyocarditis virus, Cosavirus A, Human cytomegalovirus, Human T-lymphotropic virus, Hepatitis delta virus, Adeno-associated virus, Ebolavirus, Human rhinovirus, Coxsackievirus, Echovirus, Human enterovirus, Poliovirus, Human parvovirus B19, Murray valley encephalitis virus, Dengue virus, Japanese encephalitis virus, Langat virus, Louping ill virus, St. louis encephalitis virus, Tick-borne powassan virus, West Nile virus, Yellow fever virus, Zika virus, Hantaan virus, New York virus, Puumala virus, Seoul virus, Hendra virus, Nipah virus, Hepatitis virus, Influenza virus, Aichi virus, Human immunodeficiency virus, Cercopithecine herpesvirus, Epstein-Barr virus, Australian bat lyssavirus, Duvenhage virus, Lagos bat virus, Mokola virus, Rabies virus, European bat lyssavirus, Human astrovirus, Lake Victoria marburgvirus, Human adenovirus, Molluscum contagiosum virus, Measles virus, Human papillomavirus, Crimean-Congo hemorrhagic fever virus, Dugbe virus, Norwalk virus, Southampton virus, Oropouche virus, Bunyamwera virus, Bunyavirus La Crosse, Bunyavirus snowshoe hare, Hepatitis B virus, Human respiratory syncytial virus, Monkeypox virus, Cowpox virus, Horsepox virus, Vaccinia virus, Variola virus, Yaba monkey tumor virus, Yaba-like disease virus, Orf virus, GB virus C/Hepatitis G virus, Punta toro phlebovirus, Rift valley fever virus, Sandfly fever Naples phlebovirus (Toscana virus), Sandfly fever sicilian virus, Uukuniemi virus, BK polyomavirus, JC polyomavirus, KI Polyomavirus, Merkel cell polyomavirus, WU polyomavirus, Human parainfluenza, Rosavirus, Human herpesvirus, Rotavirus, Rubella virus, Mammalian orthorubulavirus (Simian virus), Mumps virus, Salivirus A, Sapporo virus, Banna virus, Eastern chimpanzee simian foamy virus, Simian foamy virus, Dhori virus, Vientovirus, Human torovirus, Varicella-zoster virus, Chandipura virus, Isfahan virus, and Vesicular stomatitis virus.

10. The vaccine depot formulation of aspect 6, wherein the antigen is from an influenza virus, SARS-CoV-2, or Coxiella burnetii.

11. The vaccine depot formulation of any one of aspects 1 to 10, wherein the antigen is an antigenic protein, or antigenic peptide.

12. The vaccine depot formulation of any one of aspects 1 to 5, wherein the antigen is an endogenous antigen from a cancer or is associated with an immune disease.

13. The vaccine depot formulation of aspect 12, wherein the antigen is a cancer tumor-specific antigen selected from NY-ESO-1, gp100, CT26, alphafetoprotein (AFP), carcinoembryonic antigen (CEA), CA-125, CA15-3, CA19-9, MUC-1, epithelial tumor antigen (ETA), tyrosinase, melanoma-associated antigen (MAGE), abnormal products of ras or p53, CTAG1B, MAGEA1, and HER2/neu.

14. The vaccine depot formulation of any one of aspects 1 to 13, wherein the nanoparticles are hollow protein nanoparticles.

15. The vaccine depot formulation of aspect 14, wherein the hollow protein nanoparticles are based on ferritin, TIP60, or hepatitis B virus (HBV) surface-antigen protein.

16. The vaccine depot formulation of aspect 15, wherein the hollow protein nanoparticles are based on the E2 subunit, or a portion thereof, of the pyruvate dehydrogenase complex (PDC) from the Geobacillus stearothermophilus.

17. The vaccine depot formulation of aspect 16, wherein the lipoyl domain of the E2 subunit has been removed resulting in a E2 protein that comprises 256 amino acids and has the sequence of SEQ ID NO:44.

18. The vaccine depot formulation of aspect 16 or aspect 17, wherein the E2 subunit of the PDC has been recombinantly modified to substitute one or more amino acids with cysteines.

19. The vaccine depot formulation of any one of aspects 16 to 18, wherein the recombinant E2 subunit has a polypeptide sequence that is at least 95%, at least 98%, or at least 99% identical to the sequence presented in SEQ ID NO:5 and which has an amino acid substitution of D214C in comparison to SEQ ID NO:45.

20. The vaccine depot formulation of aspect 19, wherein the recombinant E2 subunit has a polypeptide sequence of SEQ ID NO:5, SEQ ID NO:7, or SEQ ID NO:9.

21. The vaccine depot formulation of aspect 19 or aspect 20, wherein the recombinant E2 subunit has a polypeptide sequence of SEQ ID NO:5.

22. The vaccine depot formulation of any one of aspects 1 to 21, wherein the adjuvant is selected from ssRNA, MGN1703, aluminum hydroxide, aluminum phosphate, and aluminum potassium sulfate, AS04, MF59, and AS01_{B}.

23. The vaccine depot formulation of any one of aspects 1 to 21, wherein the adjuvant is an CpG oligodeoxynucleotide based adjuvant.

24. The vaccine depot formulation of aspect 23, wherein the CpG oligodeoxynucleotide based adjuvant is selected from CpG1018, CpG1826, CpG ODN 1466, CpG ODN PB3, CpG ODN BW005, CpG Alum, CPG 21424, CpG 7909, CpG-ODN 2135, ODN K3, CpG ODN 10101, CpG-28, CpG ODN C274, CpG ODN C695, CpG ODN #17, CpG-ODN 2722, CpG 8916, CpG 8954, CpG ODN 678, CpG ODN BW015, CpG ODN 658, CpG ODN 640, CpG ODN PB9, CpG ODN BW004, CpG ODN BW103, CpG ODN 110, CpG ODN BW206, CpG ODN 607, CpG ODN 647, CpG ODN 111, CpG ODN 109, CpG ODN 656, CpG ODN 664, CpG ODN C9, CPG2429, CPG5475, CPG21608, CPG21797, CPG21796, CPG21799, CPG21800, CPG21802, CPG21889, CPG23409, CpG-c41, CPG23410, CPG23411, CPG23412, CPG23413, CPG23617, CPG23414, CPG 1681, CPG 2143, CPG 21425, and CPG 21426.

25. The vaccine depot formulation of aspect 23, wherein the CpG oligodeoxynucleotide based adjuvant has the sequence of SEQ ID NO:40.

26. The vaccine depot formulation of any one of aspects 1 to 25, wherein the antigen is conjugated to the outer surface of the nanoparticles, and wherein the adjuvant is conjugated or loaded into the inner cavity of the nanoparticles.

27. The vaccine depot formulation of any one of aspects 1 to 25, wherein the adjuvant is conjugated to the outer surface of the nanoparticles, and wherein the antigen is conjugated or loaded into the inner cavity of the nanoparticles.

28. The vaccine depot formulation of any one of aspects 1 to 27, wherein the antigen and/or the adjuvant are conjugated to the outer surface and an accessible inner cavity of the nanoparticles by use of mal-tNTA-Ni, sulfo-SMCC, sortase A ligation, or by use of SpyCatcher/SpyTag.

29. The vaccine depot formulation of aspect 28, wherein the antigen is conjugated to the outer surface of the nanoparticles by use of SpyCatcher/SpyTag, and wherein the adjuvant is loaded or conjugated to the inner cavity of the nanoparticles by use of a linker that binds to both the adjuvant and free cysteine groups in the inner cavity of the nanoparticles.

30. The vaccine depot formulation of aspect 29, wherein the adjuvant has been modified to comprise a terminal aldehyde or benzaldehyde group, and wherein the linker is a N-(β-maleimidopropionic acid) hydrazide (BMPH) linker.

31. The vaccine depot formulation of aspect 30, wherein the adjuvant has the sequence of SEQ ID NO:40 that comprises a terminal 5′ benzaldehyde.

32. The vaccine depot formulation of any one of aspects 29 to 31, wherein the outer surface of the nanoparticles is conjugated with a SpyTag peptide, and wherein the antigen is conjugated with a SpyCatcher peptide.

33. The vaccine depot formulation of aspect 32, wherein the SpyTag peptide has the sequence of SEQ ID NO:17, and wherein the SpyCatcher peptides has the sequence of SEQ ID NO: 19.

34. The vaccine depot formulation of aspect 32 or aspect 33, wherein the nanoparticles comprise a recombinant E2 subunit that has a polypeptide sequence that is at least 95%, at least 98%, or at least 99% identical to the sequence presented in SEQ ID NO:5 and which has an amino acid substitution of D214C in comparison to SEQ ID NO:45.

35. The vaccine depot formulation of aspect 34, wherein the SpyTag conjugated recombinant E2 protein nanoparticles (ST-E2) have the sequence of SEQ ID NO:21.

36. The vaccine depot formulation of any one of aspects 32 to 35, wherein the antigen that is conjugated with a SpyCatcher peptide is CBU1910 and the construct (SC-CBU1910) has the sequence of SEQ ID NO:23.

37. The vaccine depot formulation of any one of aspects 32 to 35, wherein the antigen that is conjugated with a SpyCatcher peptide is H5 hemagglutinin and the construct (SC-H5) has the sequence of SEQ ID NO:43.

38. A method of immunizing a subject comprising:
administering one or more doses of the vaccine depot formulation of any one of aspects 1 to 37 to protect a subject from an infection by a pathogen and/or from a disease.

39. The method of aspect 38, wherein the infection and/or disease is selected from Q fever, avian influenza, and SARS-CoV-2.

40. The method of aspect 38 or aspect 39, wherein the vaccine depot formulation is formulated for intramuscular administration, subcutaneous administration, or intravenous administration.

41. The method of any one of aspects 38 to 40, wherein the vaccine depot formulation provides long-lasting, sustained delivery of the antigen for at least 2, 4, 6, 8, 10, or 12 weeks in vivo.

42. A method to make the vaccine depot formulation of any one of aspects 1 to 37, comprising:
reacting SpyTag conjugated recombinant E2 protein (ST-E2) nanoparticles comprising a plurality of free cysteine groups with a linker and adjuvant comprising a terminal aldehyde or benzaldehyde group, to form ST-E2 nanoparticles comprising the adjuvant; and
reacting the ST-E2 nanoparticles comprising the adjuvant with a SpyCatcher conjugated antigen to form the vaccine depot formulation.

43. The method of aspect 42, wherein the ST-E2 nanoparticles have the sequence of SEQ ID NO:21.

44. The method of aspect 42 or 43, wherein the linker is a N-(β-maleimidopropionic acid) hydrazide (BMPH) linker.

45. The method of any one of aspects 42 to 44, wherein the adjuvant has the sequence of SEQ ID NO:40 that comprises a terminal 5′ benzaldehyde.

46. The method of any one of aspects 42 to 45, wherein the SpyCatcher conjugated antigen comprises a foreign antigen from a pathogen or an endogenous antigen from a cancer or is associated with an immune disease.

47. The method of any one of aspects 42 to 46, wherein a SpyCatcher peptide that has the sequence of SEQ ID NO:19 is conjugated to the antigen.

The following examples are intended to illustrate but not limit the disclosure. While they are typical of those that might be used, other procedures known to those skilled in the art may alternatively be used.

### Examples

Materials. Chemical reagents are purchased from Fisher Scientific, Sigma-Aldrich, Acros Organics, Iris Biotech, or TCI Pharmaceuticals unless otherwise noted. Phosphate buffer used in the reactions in this study comprised 50 mM KH₂PO4 and 100 mM NaCl at a pH 7.4. Phosphate-buffered saline (PBS) used for in vivo studies is purchased from Gibco. All cloning enzymes are purchased from New England Biolabs (NEB), unless otherwise noted. DH5α and BL21(DE3) E. coli are used for general cloning and expression studies, respectively. DNA minipreps and gel extractions are performed with the QIAprep Spin Miniprep Kit (Qiagen) and GeneJET Gel Extraction Kit (Thermo Fisher Scientific), respectively. DNA primers are synthesized and ordered from Integrated DNA Technologies (IDT). CloneJET PCR cloning kit (Thermo Fisher Scientific) is used for all polymerase chain reactions (PCRs). Plasmid pET11a is used as the expression vector for all protein constructs.

Protein Microarrays. Protein microarrays were fabricated as previously described Hernandez-Davies et al., Frontiers in Immunology 12(18):692151 (2021), Briefly, antigenic protein or peptide (e.g., CBU1910 or H5) is diluted to a concentration of 0.1 mg/mL and printed onto nitrocellulose-coated glass Oncyte Avid slides (Grace Bio-Laboratories) using an Omni Grid 100 microarray printer (Genomic Solutions). For probing, mouse plasma samples are diluted 1:100 in protein array blocking buffer supplemented with 10 mg/mL E. coli lysate (GenScript). Arrays are rehydrated with blocking buffer prior to addition of preincubated sera. Arrays are incubated overnight at 4° C. with gentle agitation. After overnight incubation, the slides are washed with Tris-buffered saline (TBS) containing 0.05% Tween 20 (T-TBS) and incubated with biotinylated-SP-conjugated goat anti-mouse IgG, IgG1, or IgG2c (Jackson Immunoresearch). Arrays are washed with T-TBS and incubated with streptavidin conjugated Qdot-800 (ThermoFisher). Arrays are washed three times with T-TBS followed by TBS, dipped in water, and dried by centrifugation. Images were acquired using the ArrayCAM imaging system (Grace Bio-Laboratories). Spot and background intensities are measured using an annotated grid (.gal) file. IgG1 and IgG2c antibody subtype proportions are calculated using respective signal intensities: IgG1/(IgG1+IgG2c) and IgG2c/(IgG1+IgG2c), respectively.

Mice and Immunizations. All animal studies are carried out in accordance with protocols approved by the Institutional Animal Care and Use Committee (IACUC) at the University of California, Irvine. Briefly, 6-8-week-old female C57BL/6 mice (n=5) are immunized subcutaneously at the left flank on Day 0 and followed by a booster on Day 14. Injections were 30 μL per mouse and contained definite amounts of a model antigen, E2, and CpG, based on the formulations investigated. In groups that used the adjuvant, IVAX, an equal volume of IVAX to formulation is supplemented (i.e., 30 μL of E2 formulation+30 μL IVAX). IVAX contains Addavax (InvivoGen), 1 nmol of CpG 1018, and 3 nmol of MPLA. Seven days after the last immunization, mice are sacrificed, blood is collected via cardiac puncture, and spleens are isolated.

T Cell Recall Assays. Recall assays are performed using IFN-γ ELISpot format and spleens collected on day 21 essentially as described in Hernandez-Davies et al., Sci. Rep 12(1):9198 (2022). Antigens used for recall are avian influenza H5, C. burnetii CBU1910 or OVA as an irrelevant control antigen. Assays are performed in RPMI 1640 media, containing 5×10⁻⁵ M β-mercaptoethanol, 100 IU/mL penicillin, 100 μg/mL streptomycin, and 10% heat-inactivated fetal bovine serum (complete medium). Briefly, erythrocyte-depleted splenocytes are incubated at 5×10⁵ cells per well in 96-well ELISpot plates, coated previously with IFN-γ capture antibody, and blocked in complete medium, containing titrations of antigen ranging from 2.5 to 10 μg/mL. Mice are assayed separately. Concanavalin A is included as a viability control.

Statistical Analysis. For nanoparticle characterization, including hydrodynamic diameter measurements, molecular weights determined by mass spectrometry, and antigen/nanoparticle ratios, data are presented as the mean±standard deviation (S.D.) of at least three independent experiments (n≥3), unless otherwise noted. Statistical analysis of immunization data was carried out by using GraphPad Prism. Data are presented as mean±standard error of the mean (S.E.M.) from at least five independent individuals (n≥5). Statistical analysis was determined by a one-way or two-way ANOVA over all groups, followed by a Bonferroni multiple comparison test, unless otherwise noted. P-values less than 0.05 were considered significant.

E2 Nanoparticle Expression and Purification. In summary, cells are grown in LB broth containing 100 μg/mL ampicillin and induced at OD600 0.7 to 0.9 with 1 mM IPTG at 37° C. for 3.5 h. Purification is performed using a Q-Sepharose anion exchange column and a Superose 6 size exclusion column (GE Healthcare), with the exception that 1 mM DTT is added to all of the lysis, purification, and storage buffers to prevent cross-linking of protein nanocapsules in solution. Proteins are stored at 4° C. for short-term or −80° C. for long-term storage in 50 mM potassium phosphate (pH 7.4) and 100 mM NaCl (herein referred to as “phosphate buffer”) containing 0.02% sodium azide, 1 mM DTT, and 5 mM EDTA. Protein concentrations are quantified using a Micro-BCA kit (Pierce). To enable chemical attachment of non-native molecules to the surface of the protein nanocapsules, several potential external sites were identified to perform site-directed mutagenesis to cysteine. The thiol side chain of cysteine is advantageous because it enables chemical conjugation at specific sites constructed via protein engineering.

Construction of CBU1910-E2 Fusion Protein Mutants. Previously established E2 mutants E2_152 and E2_158 are used to engineer CBU1910-E2 fusion constructs. D381C is an E2 mutation that introduces 60 cysteines to the internal cavity of the nanoparticle, allowing for internal conjugation. To introduce the D381C mutation to E2_158 and E2_152 via site directed mutagenesis (SDM) the forward primer: 5′-/5Phos/GCCGATCGTTCGTTGCGGTGAAATCGTTGC-3′ (SEQ ID NO:24) and reverse primer: 5′-/5Phos/TTTTCGGCTATACGACCAATACCCAG-3′ (SEQ ID NO:25) are used. To introduce the DNA cut sites required for ligation to the N-terminus of E2 mutants, Nde1 and Nhe1 cut sites are introduced to the N-terminus DNA coding region and C-terminus DNA coding region, respectively, of CBU1910 using the forward primer: 5′-CATATGCACCATCACCATCACCATCCGCAGCAAGTCAAAGACATTCAG-3′(SEQ ID NO:26) and reverse primer: 5′-GCTAGCTTAGCCGCCGGTTTCCGG-3′ (SEQ ID NO:27) The plasmid encoding the CBU1910 protein (with its signal peptide deleted and portion of N-terminus truncated) was previously synthesized by GenScript Biotech and is used as the DNA template for all genetic engineering of the protein antigen.

A standard Phusion High-Fidelity DNA polymerase protocol is used for PCRs. These reactions are performed in a thermal cycler using a 30 s denaturation step at 98° C., followed by 30 cycles of 15 s at 98° C., 15 s at 58° C. (E2 D381C mutation) or 53° C. (CBU1910), and 7 min (E2 D381C mutation) or 45 s (CBU1910) at 72° C., with a final step of 10 min at 72° C. The CBU1910 gene is then ligated via the Nde1/Nhe1 sites of a pET11a vector that contained the E2 gene between Nhe1/BamH1.

E2 (E297C) is an E2 mutant that displays 60 cysteines on its surface that can be used for thiol-based functionalization. Purified E2 (E279C) in 20 mM HEPES and 100 mM NaCl (pH 7.3) is incubated with an 8.5× molar excess of TCEP (Thermo Fisher Scientific; dissolved in Milli-Q water). A 10× molar excess of maleimido cyclic tris-NTA (mal-tNTA) (diluted to 4 mg/mL in DMF) is added to the E2 and incubated at room temperature for 2 h and then at 4° C. overnight. Unreacted mal-tNTA, DMF, and TCEP are removed using Zeba spin desalting columns in 20 mM HEPES and 100 mM NaCl. Conjugation efficiency and characterization are determined using SDS-PAGE and mass spectrometry (Xevo G2-XS QTof). The hydrodynamic diameter of the purified constructs was analyzed by dynamic light scattering (DLS) (Malvern Zetasizer Nano ZS).

Loading C. burnetii peptide antigen onto E2 nanoparticles. Given the desire to elicit a robust adaptive immune response towards the pathogen of C. burnetii, peptide antigen epitopes are conjugated to the E2 nanoparticle. Using a CBU1910 peptide, HYLVNHPEVLVEASQ (CBUl910p) (SEQ ID NO:28), that has been shown to be a T cell specific epitope, the CBU1910p-CpG-E2 formulation is synthesized. Optimization of the peptide conjugation is important for synthesizing well loaded and stable constructs. To increase its physical stability and prevent aggregation, the final formulation contained 0.005% (v/v) Tween 20. Conjugation of CBU1910p is supported by ˜1.8 kDa incremental increases to E2 monomer molecular weight seen on SDS-PAGE. Using a BMPH linker, aldehyde modified CpG is conjugated in the core of the E2 nanoparticle. Loading of CpG was confirmed by a ˜7 kDa increase in E2 monomer molecular weight from ˜28 kDa to ˜35 kDa. SDS-PAGE and mass spectrometry were used to determine the loading of CBU1910p and CpG per nanoparticle. Quantification indicated 22.3±1.5 CpG 1826 molecules were conjugated internally and 166±11.2 CBU1010p peptides are conjugated externally per 60-mer E2 nanoparticle, similar to previous E2 formulations. The average hydrodynamic diameter of CpG-E2, CBU1910p-E2 and CBU1910p-CpG-E2 nanoparticles was 27.1±0.4 nm, 35.4±2.7 nm, and 31.9±1.7 nm, respectively.

CpG conjugation to E2 nanoparticles. Aldehyde-terminated CpG oligonucleotides (e.g., CpG1826) were covalently packaged within E2. Briefly, the cysteines in the E2 internal cavity are reduced with TCEP (Pierce), followed by incubation with N-(β-maleimidopropionic acid) hydrazide (BMPH) linker (Pierce) and removal of unreacted linker. Conjugation with the aldehyde-modified CpG 1826 involved overnight incubation and excess CpG removal. The number of conjugated CpG molecules is determined previously to average 22 CpG molecules per E2 particle; this conjugation ratio is kept constant throughout this study.

Construction of SpyTag-E2 Mutants and SpyCatcher-CBU1910 Fusion Protein. The SpyTag/SpyCatcher system was used to attach CBU1910 to the E2 nanoparticle. The advantages of this approach include its stable covalent interaction and the ability to separately express both the antigen and the nanoparticle proteins prior to conjugation, which can circumvent protein expression challenges. SpyTag (ST) is genetically attached to the E2 nanoparticle, and SpyCatcher (SC) is genetically fused with the protein antigen CBU1910. It was postulated that coupling SpyCatcher to the antigen minimizes the amount of exposed SC after conjugation to the NP, which is likely favorable for reducing anti-SC immune responses.

Previously established mutants E2(D381C) and E2_152 were used to engineer the SpyTag-E2 platforms. To introduce the D381C mutation to E2_152 via site directed mutagenesis (SDM) the forward primer: 5′-/5Phos/GCCGATCGT TCGTTGCGGTGAAATCGTTGC-3′ (SEQ ID NO:29) and reverse primer: 5′-/5Phos/TTT TCGGCTATACGACCAATACCCAG-3′ (SEQ ID NO:30) were used. Introduction of the SpyTag to E2(D381C) and E2_152 was done using the forward primers: 5′-CATATG GCCCACATCGTTATGGTGGATGCCTACAAGCCAACTAAA GGT TCAGGAAC AGCAGGTGGTGGGTCAGGTTCCCTGTCTGTTCCTGGTCCCGC-3′ (SEQ ID NO:31) and 5′-CATATGGCCCACATCGTTATGGTGGATGCCTACAAGCCAA CT AAAGCTAGCACCGGCAAAAATGGTCG-3′ (SEQ ID NO:32), respectively. E2 mutants used the same reverse primer: 5′-GGATCCTTAAGCTTCCATCAGCAGCAGTTCC GG-3′ (SEQ ID NO:33).

The plasmid encoding the truncated CBU1910 protein is previously synthesized by GenScript Biotech. The plasmid containing the SpyCatcher gene (pDEST14-SpyCatcher) was obtained from Addgene. To introduce the endonuclease sites and GS-rich spacer on CBU1910 for fusion to SpyCatcher, the forward primer was 5′-GCTAGCG GTTCAGGAACAGCAGGTGGTGGGTCAGGTTCCCCGCAGCAAGTCAAAGACATTC-3′ (SEQ ID NO:34) and the reverse primer was 5′-GGATCCTTATTTTTCGACACGGTCA ATTTCTTTTTGCAGG-3′ (SEQ ID NO:35). To introduce the endonuclease sites on SpyCatcher, the forward primer 5′-CATATGTCGTACTACCATCACCATCACCATCACG-3′ (SEQ ID NO:38) and reverse primer 5′-GCTAGCAATATGAGCGTCACCT TTAGTTGCTTTGCC-3′ (SEQ ID NO:39) are used. A standard Phusion High-Fidelity DNA polymerase protocol was used for PCRs. These reactions are performed in a thermal cycler using a 30 s denaturation step at 98° C., followed by 30 cycles of 15 s at 98° C., 15 s at 56° C. (SpyTag introduced to E2) or 55° C. (SpyCatcher) or 52° C. (CBU1910), and 45 s (SpyTag introduced to E2) or 40 s (SpyCatcher) or 45 s (CBU1910) at 72° C., with a final step of 10 min at 72° C.

Expression, Purification, and Characterization of SpyTag-E2 Particles. E2 protein mutants are prepared similarly to previously described mutants in Molino et al., ACS Nano 7(11):9743-9752 (2013), Molino et al., Biomacromolecules 13:974-981 (2012), and Neek et al., Biomaterials 156:194-203 (2018). Mutant ST-E2(D381C) was ultimately chosen for scale up expression. Briefly, a 1 L culture supplemented with 100 μg/mL of ampicillin is inoculated with an overnight culture at 37° C. until an OD of 0.7-0.9 at which time it is induced by 1 mM IPTG and further incubated for 3 h at 37° C. Cells are pelleted and stored at −80° C. overnight before breaking. Cells are lysed using a lysing buffer containing 3 mM PMSF and French Press (Thermo Fisher Scientific). Soluble cell lysates are heat shocked at 70° C. and ultracentrifuged to remove thermolabile containments. Subsequently, the lysates are purified using a HiPrep Q Sepharose anion exchange column (GE Healthcare) followed by a Superose 6 prep grade (GE Healthcare) size exclusion column. The purified proteins are characterized by DLS (Zetasizer Nano ZS, Malvern), mass spectrometry (Xevo G2-XS QTof) and SDS-PAGE, and bicinchoninic acid assay (BCA) for size, molecular weight and purity, and protein concentration, respectively.

The residual E. coli expression derived lipopolysaccharide (LPS) is removed. Briefly, Triton X-114 (Sigma) is added to the purified protein at 1% (v/v), chilled to 4° C., vortexed vigorously, and heated to 37° C. The mixture is then centrifuged at 18000×g and 37° C. for 1 min, and the protein-containing aqueous phase is separated from the detergent phase. This total process is repeated 9 times. Residual Triton is removed with detergent removal spin columns (Pierce). LPS levels are tested to be below 0.1 EU per microgram of E2 protein (LAL ToxinSensor gel clot assay, Genscript).

Expression, Purification, and Characterization of SpyCatcher-CBU1910 The SpyCatcher-CBU1910 fusion protein is expressed in a fashion similar to that of the E2 particles. Proteins are expressed in E. coli via 1 mM IPTG induction. After induction for 3 h at 37° C., the cells are pelleted and stored at −80° C. before breaking. Cells are lysed via French Press and soluble protein is purified using a HisPur Ni-NTA resin batch protocol (Thermo Fisher Scientific). Briefly, soluble cell lysates were mixed with equal parts equilibration buffer and applied to a HisPur Ni-NTA affinity spin column using a packing ratio of 1.5 mL of resin per 10 mL of lysate slurry. The lysate was allowed to incubate with the resin for 1 h at 4° C. Wash buffers and elution buffer containing 75 and 150 mM imidazole, and 250 mM imidazole, respectively, were used to attain pure SC-CBU1910. Pure protein fractions were collected and dialyzed into PBS to remove imidazole using 6-8 kDa MWCO dialysis tubing. The purified protein was characterized by mass spectrometry (Xevo G2-XS QTof) and SDS-PAGE, and BCA for molecular weight and purity, and protein concentration, respectively.

Residual E. coli expression derived LPS was removed in a similar fashion to the E2 protein. Residual Triton was removed with detergent removal spin columns (Pierce) or SM2 detergent removal beads (Bio-Rad). LPS levels are below 0.1 EU per microgram of SC-CBU1910 protein (LAL ToxinSensor gel clot assay, Genscript).

CpG and SpyCatcher Conjugation onto SpyTag-E2 Particles. The oligodeoxynucleotide TLR-9 ligand CpG 1826 (5′-TCCATGACGTTCCTGACGTT-3′) (SEQ ID NO:40) (CpG) was synthesized with a phosphorothioated backbone and 5′ benzaldehyde modification by Integrated DNA Technologies (IDT). CpG is conjugated to the internal cavity of the E2 nanoparticle as described in Molino et al., ACS Nano 7(11):9743-9752 (2013). In brief, the 60 internal cavity cysteines of E2 are reduced with TCEP (Pierce) for 30 min, followed by incubation with the N-(β-maleimidopropionic acid) hydrazide (BMPH) linker (Pierce) for 2 h at room temperature (RT). Unreacted linker is removed using 40 kDa cutoff Zeba spin desalting columns (Pierce). The aldehyde-modified CpG is subsequently added and incubated overnight at RT. Unreacted CpG is removed by desalting spin columns. Conjugation is estimated by SDS-PAGE and measured by band intensity analysis.

As diagramed in FIG. 3, directly incubating SpyCatcher-CBU1910 and SpyTag-E2 particles will allow for spontaneous isopeptide bond formation and conjugation. SC-CBU1910 proteins are incubated with ST-E2 particles at a ˜0.5:1 (SC-CBU1910:ST-E2 monomer) molar ratio, supplemented with 0.080-0.0875% (w/v) Sarkosyl (SLS), for 20 h at room temperature. SDS-PAGE densitometry analysis with protein standards is used to quantify protein loading onto the particles.

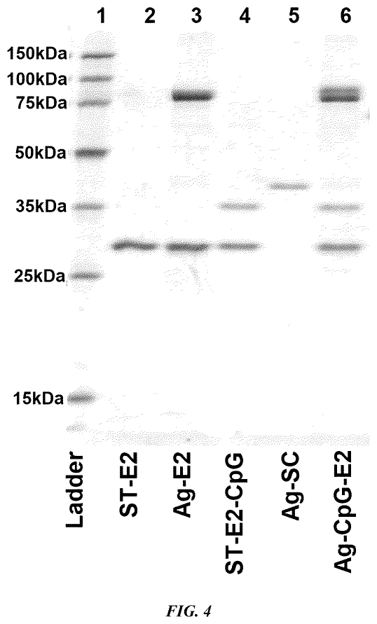

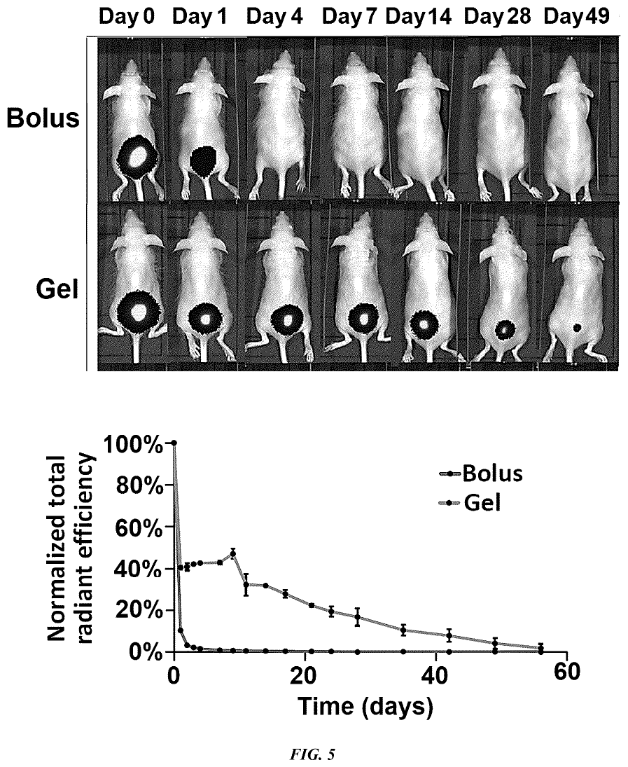

Characterization of ST-E2 nanoparticles that conjugated with a model antigen (i.e., CBU1910) and with a CpG adjuvant. The nanoparticles were characterized with SDS-PAGE and the amount of CpG and antigen on each nanoparticle was quantified. On each E2 nanoparticle, there are 20.5±1.5 CpG molecules on internal cavity and 24±2 antigens on the surface (e.g., see FIG. 4).

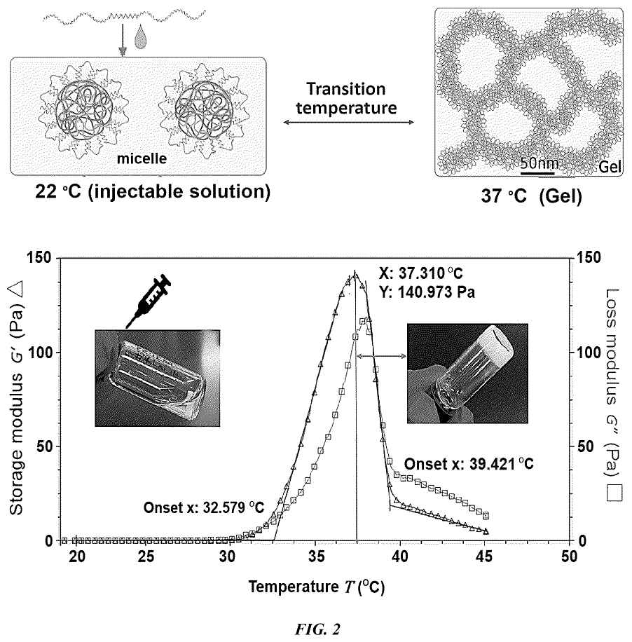

Preparation of the hydrogel depot material. The hydrogel depot material can be easily prepared by dissolving the block polymer in PBS. It is a liquid at room temperature and can be loaded with the antigen and adjuvants by simply mixing. It is injectable under room temperature. After injected and temperature rise above 32.6° C., it turns into a hydrogel (e.g., see FIG. 2).

Using SpyTag(ST)/SpyCatcher(SC) to Conjugate CBU1910 onto E2 Nanoparticles. The ST peptide is genetically fused to the N-terminus of E2 with a spacer sequence. The E2 mutant D381C possessed 60 internal cavity cysteines, which would enable conjugation of adjuvant. Because a high-resolution protein structure of CBU1910 has not yet been determined, the protein folding prediction tool Alphafold2 is used to predict the structure of CBU1910. Based on this predicted structure of N-terminal truncated CBU1910 (to enable a soluble antigen), it was decided to fuse SC to the N-terminus of CBU1910. This ensured that when conjugated to the E2 nanoparticle, CBU1910 would be oriented in the same direction as when it is displayed on C. burnetii, exposing more relevant B cell epitopes.

The attachment of ST and SC to E2 and CBU1910, respectively, did not appear to decrease the expression levels or soluble protein amounts. Therefore, ST-E2(D381C) (henceforth referred to as ST-E2) and SC-CBU1910 are purified for further characterization and studies. Both SDS-PAGE and mass spectrometry shows an expected molecular weight increase of ˜2.2 kDa (ST and spacer) for ST-E2 monomers. The ST-E2 NP assembly yielded a hydrodynamic diameter of 29.2±0.5 nm, which is slightly larger than the E2 diameter size of 27.8±0.6 nm, as expected. This is approximately 1 nm larger, which is consistent with previous literature estimates of ST on virus-like particles (VLPs). For SC-CBU1910, a >95% purity is achieved and the average molecular weight of ˜40.8 kDa, as determined by SDS-PAGE and mass spectrometry, which is consistent with SC fused to CBU1910 with a linker.

The E2 protein NP platform, together with the ST/SC conjugation system, allows interior and exterior attachments designed for co-delivery of adjuvants and antigens, respectively. The TLR-9 agonist, CpG1826, was conjugated to the interior of the ST-E2 NP platform via an acid-labile BMPH linker.

Although it is well-documented that the isopeptide bond formation between SpyTag and SpyCatcher is robust and reliable, conjugation of the SC-CBU1910 antigen onto the surface to ST-E2 needed optimization to yield intact and monodisperse nanoparticles. As seen with other ST/SC VLP formulations, adjustments to reaction molar ratios, pH, ionic strength, and/or detergent concentrations are usually required to prevent precipitation/aggregation. The most favorable reaction conditions were found to be a 1:0.5 molar ratio of ST-E2 (monomer):SC-CBU1910 at room temperature for 20 h with the addition of 0.08-0.0875% (w/v) SLS; this resulted in stable, monodisperse nanoparticles.

Size and antigen-to-nanoparticle ratios are then determined for the CBU1910 antigen (Ag). When conjugated to SC-Ag, the ST-E2 monomer molecular weight increases by ˜41 to ˜71 kDa. As expected, when SC-Ag is conjugated to CpG-ST-E2, two conjugate bands appear: one band is Ag-E2 monomers (˜71 kDa) and the other AG-CpG-E2 monomers (˜78 kDa; both CBU1910 antigen and CpG conjugated onto E2). Quantification estimated that 29±2 and 24±2 SC-Ag are conjugated to each ST-E2 and CpG-ST-E2 nanoparticle, respectively, out of a maximum possible number of 30 per nanoparticle (based on 1:0.5 molar ratio by monomer). Ag-E2 and Ag-CpG-E2 hydrodynamic diameters were 37.9±1.9 nm and 43.6±5.1 nm, respectively, with the size increase corresponding to the successful loading of antigens on the nanoparticles. Furthermore, TEM images confirmed intact monodisperse nanoparticles. Because the ST/SC protein-protein conjugation system could be implemented to co-deliver protein antigen and adjuvant simultaneously, these stable nanoparticles (e.g., Ag-CpG-E2, Ag-E2) are used to evaluate their prophylactic vaccine potential.

The IgG responses obtained with CpG-E2-based formulations are comparable to the positive control oil-in-water emulsion adjuvant, IVAX, a combination adjuvant consisting of AddaVax (a squalene-based adjuvant), monophosphoryl-lipid A (MPLA), and CpG1018, that has been shown to induce broadly reactive responses to influenza HA proteins. Previous in vitro studies using CpG-E2 nanoparticles demonstrated that once taken up by a cell, encapsulated CpG can be released from the nanoparticle in an acidic environment and activate mouse bone marrow-derived dendritic cells. Furthermore, encapsulated CpG is shown to activate these cells at significantly lower concentrations than unbound CpG, indicating the need for bioconjugation of CpG to the nanoparticle. Other studies have also shown that alternative types of nanoparticles which simultaneously deliver both conjugated CpG and antigen can increase the immunogenicity and immune response mounted against the target antigen. Thus, to deliver adjuvants more precisely to immune cells involved in adaptive immunity, such as dendritic cells, CpG was encapsulated within the nanoparticle in an approach that increases uptake efficiency of CpG and the dose of CpG that an individual cell receives upon endocytosing a nanoparticle versus free unbound CpG. The effects of CpG adjuvant and its delivery covalently encapsulated within the E2 NP (Ag-CpG-E2) or co-administered with the E2 NP by mixing only (Ag-E2+CpG) showed comparable results on total IgG responses. Addition of IVAX to the Ag-CpG-E2 formulation further increased the overall anti-Ag IgG response.

In vivo antigen release study from the vaccine depot preparations. An in vivo release test was performed in mice to examine the retention time of antigen in the injection site when antigens are embedded in a hydrogel matrix or without hydrogel matrix. Fluorescently labeled E2 NPs were solubilized in PBS or pre-gel polymer solution and injected subcutaneously to the lower back of mice. The total radiant efficiency was monitored with IVIS. After a burst release on day 1, the gel group had about 40 percent of E2 left in the injection site and the release continued until week 8, whereas the bolus group finished releasing on day 4 (e.g., see FIG. 5).

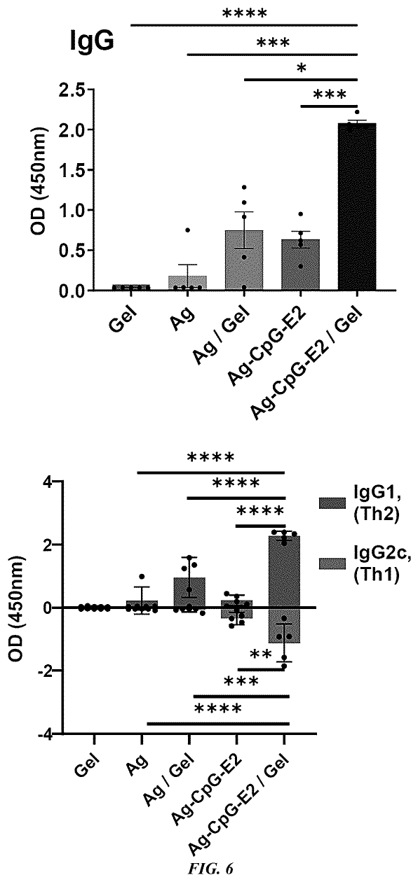

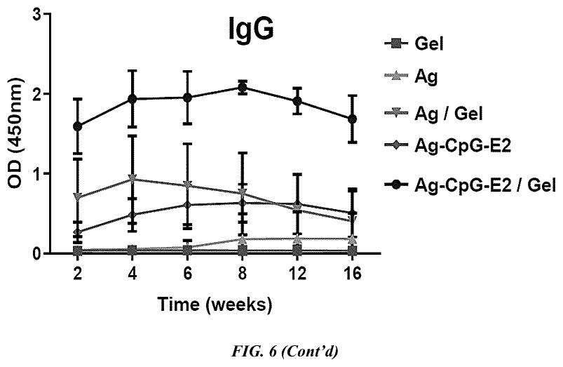

Immune response study in mice using the vaccine depot preparations. Mice immunized with Ag-CpG-E2 nanoparticles with hydrogel elicited a significantly higher IgG, IgG1, and IgG2c than the control groups with free antigen, the free antigen in hydrogel, and the Ag-CpG-E2 without hydrogel. Up to week 16 after vaccination, antigen-specific IgG of Ag-CpG-E2/Gel group is still higher than the control groups, which shows a great potential of this vaccine to provide a long-term protection. The Ag-CpG-E2 with hydrogel group also showed a more balanced IgG1/IgG2c ratio when compared with the control groups (e.g., see FIG. 6).

Conclusions from the vaccine depot preparation with the CBU1910 antigen. In the studies, a thermally sensitive hydrogel was used as a depot to slowly deliver a protein nanoparticle-based vaccine. The hydrogel material was injectable at room temperature and then gels as the temperature is increased to body temperature. In was postulated that immobilizing an antigen on a nanoparticle would enhance dendritic cell activation and antigen presentation. A model antigen (Ag) from Coxiella burnetti was conjugated onto E2 nanoparticle with adjuvant CpG 1826 (CpG) and the immunological effects of controlled nanoparticle release were investigated. The final vaccine nanoparticle complex with antigen and adjuvant (Ag-CpG-E2) was approximately 30 nm in diameter, which is within the optimal DC-uptake size range. Using an IVIS in vivo imaging system, it was shown that the slow release of fluorescently labeled nanoparticles from the hydrogel depot material lasted up to 8 weeks (e.g., see FIG. 6). Furthermore, stronger IgG, IgG1, and IgG2c responses were elicited after immunization with the hydrogel embedded with Ag-CpG-E2 than with the free antigen, the free antigen embedded in hydrogel, and the Ag-CpG-E2 without hydrogel (e.g., see FIG. 6). The Ag-CpG-E2 with hydrogel group also showed a more balanced IgG1/IgG2c ratio when compared with the control groups (e.g., see FIG. 6). The results validate the use of vaccine depot formulations or preparations for vaccination-based applications.

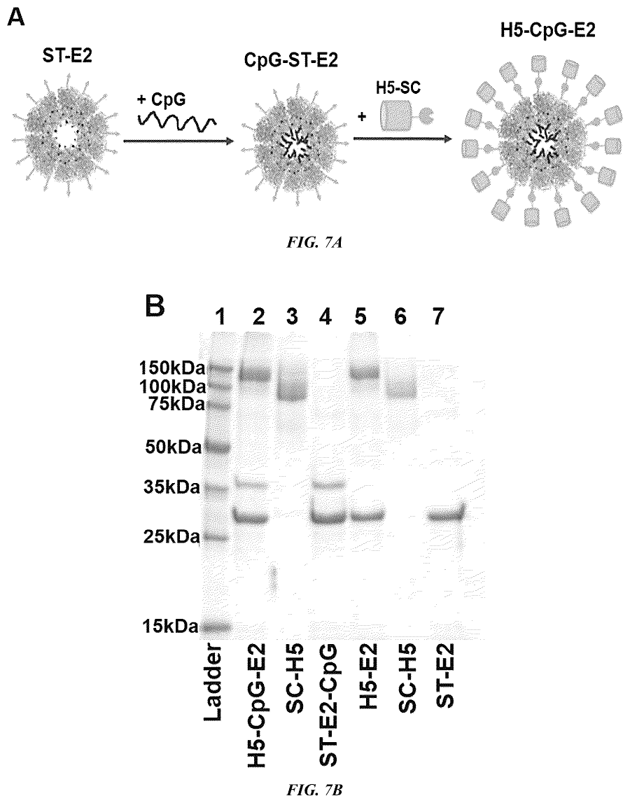

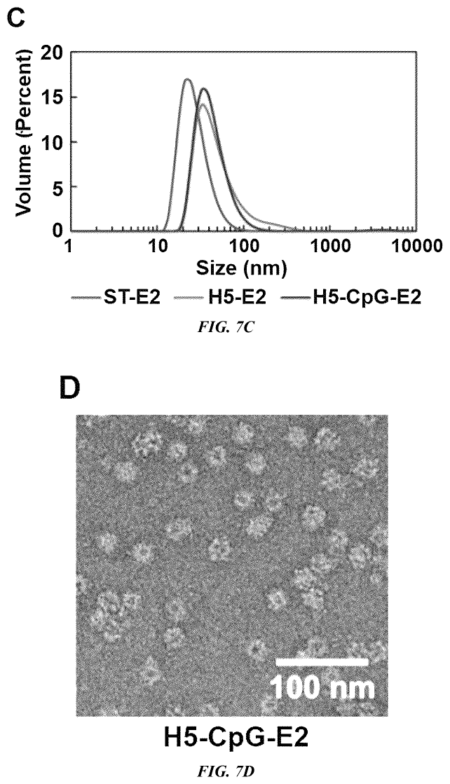

Syntheses and characterization of H5-E2 and H5-CpG-E2 nanoparticles. Based upon the foregoing results with the CBU1910 antigen, additional experiments were performed with a different antigen (i.e., H5 or H7 from avian influenza A). CpG and H5 are conjugated on E2 nanoparticles with the SpyTag-SpyCatcher reaction using a similar process described above for Ag-CpG-E2 nanoparticles. (e.g., see FIG. 7A). The structural integrity of the NP remained intact after conjugation. About 18 units of H5 molecules can be conjugated on ST-E2 to form H5-E2 nanoparticles, and about 14.8 units of CpG and 17.1 units of H5 molecules can be conjugated onto ST-E2 to form H5-CpG-E2 nanoparticles (e.g., see FIG. 7B).

The average hydrodynamic diameter of the final H5-E2 NP was found to be ˜49 nm and for H5-CpG-E2 it was found to be around ˜47 nm based on the DLS measurement (e.g., see FIG. 7C-D).

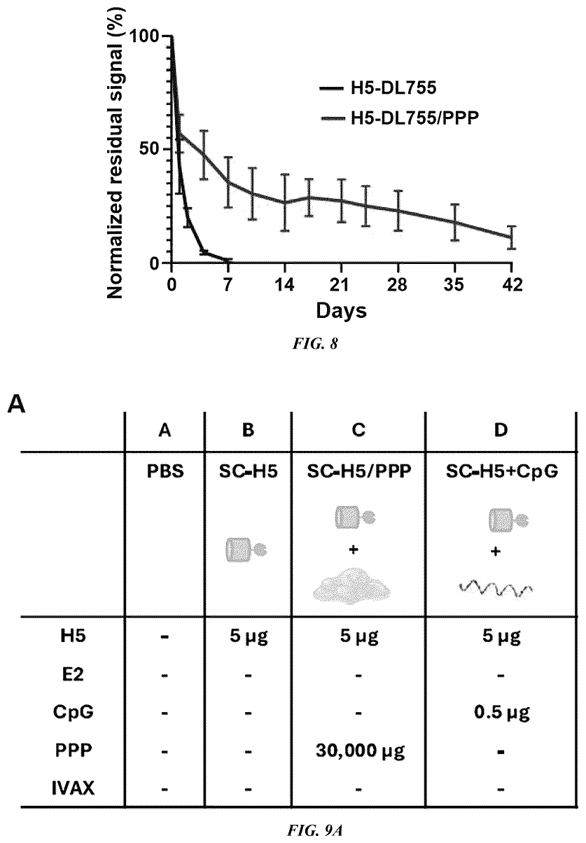

PPP hydrogel can be used as a depot material to release protein antigens. In vivo studies were performed to track the release of H5 from the constructs, with or without the hydrogel PPP, using the fluorescent label DayLight 755 (DL 755). It was found that the H5-DL755/PPP hydrogel group slowly released protein from the injection site in mice for up to 42 days, while H5-DL755 group rapidly released protein from the injection site (e.g., see FIG. 8).

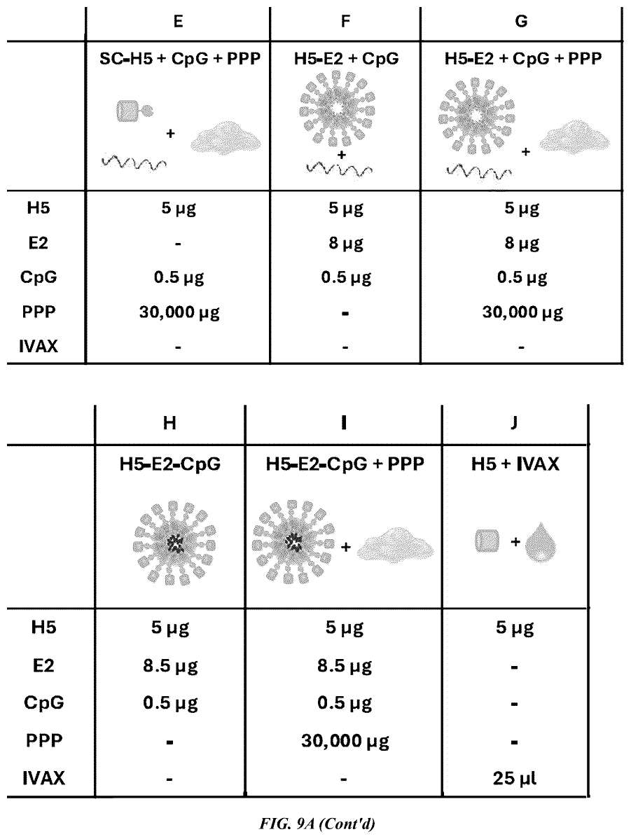

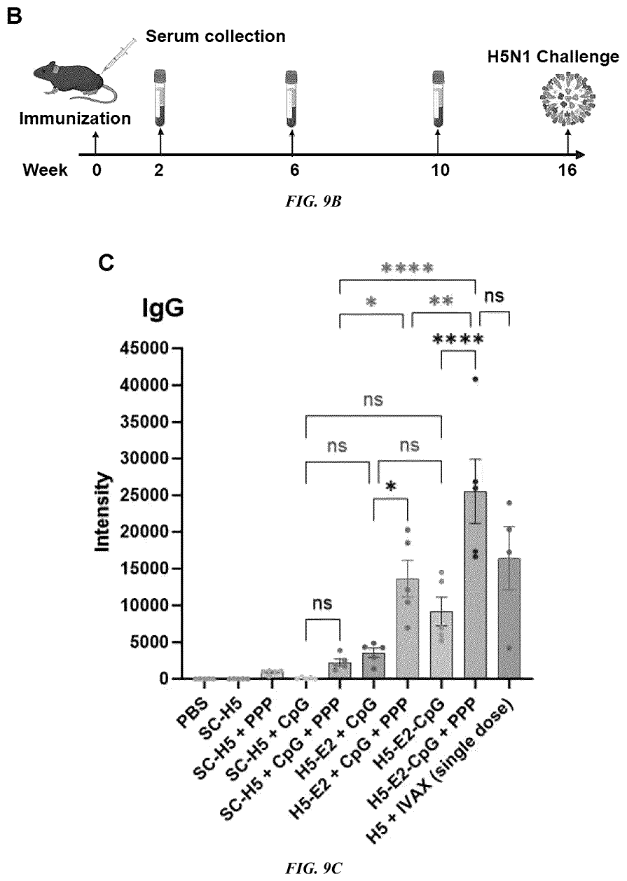

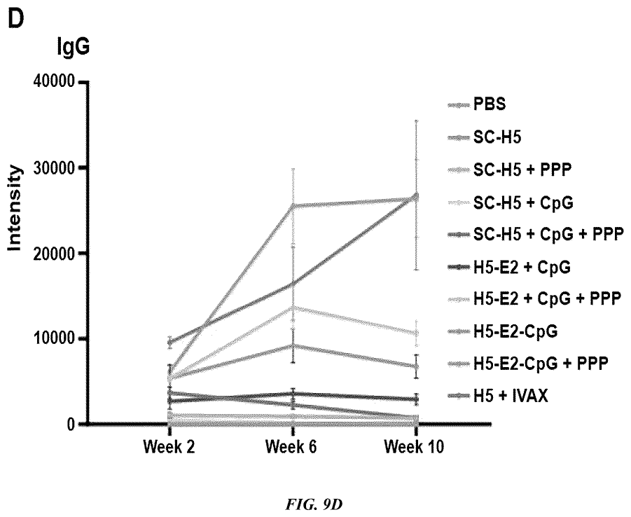

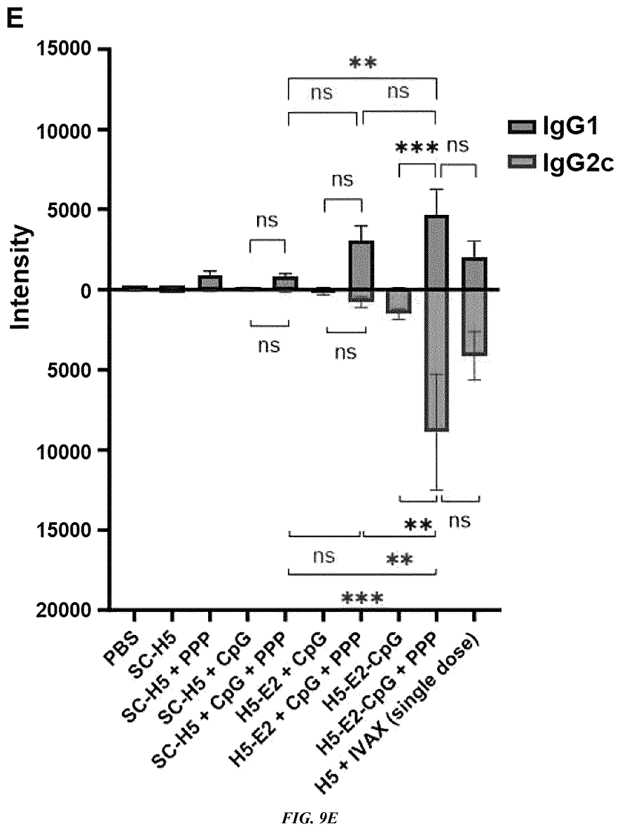

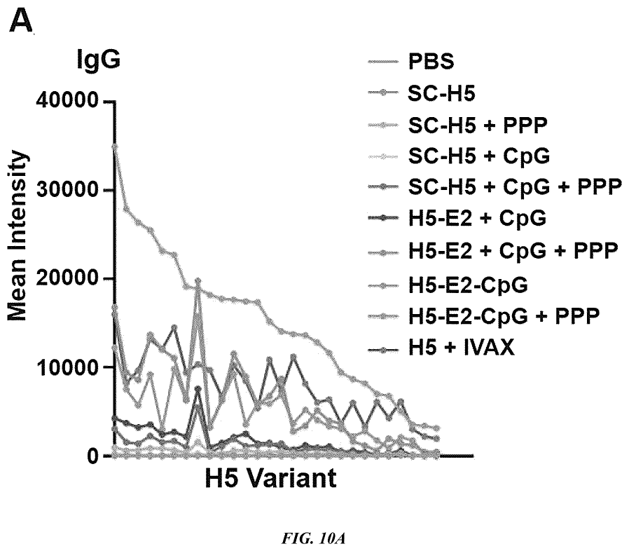

Antibody response elicited by H5-CpG-E2 loaded hydrogel vaccine against the vaccinated H5 variant. Various formulations were generated for vaccinating mice (e.g., see FIG. 9A). The following immunization schedule was followed: at week 0 the mice were immunized, serum is then collected at 2 weeks, 6 weeks, and at 10 weeks, and at week 16, the mice were challenged with HGN1 (e.g., see FIG. 9B). It was found that PPP hydrogel facilitated the increase in total IgG when H5 was conjugated on E2, no matter whether CpG was conjugated on E2 or not (e.g., see FIG. 9C, top line). Additionally, conjugating CpG and H5 on E2 was found to help increase the total IgG when loaded in PPP depot. Without the presence of PPP, conjugating H5 and CpG on E2 did not increase the total IgG significantly. H5-E2-CpG reached a higher IgG level at an earlier time point at week 6 than the H5+IVAX group and maintained the high IgG level to week 10 (e.g., see FIG. 9D). It was further found that: the PPP hydrogel facilitated the increase in both IgG1 and IgG2c when H5 and CpG were conjugated on E2; conjugating both CpG and H5 on E2 helped to increase both IgG1 and IgG2c; and conjugating CpG on E2 promoted the increase of IgG2c but not IgG1 when the vaccine was loaded in PPP (e.g., see FIG. 9E). When the vaccine was not loaded in the PPP gel, conjugating CpG and H5 did not promote the increase of IgG1 or IgG2c significantly.

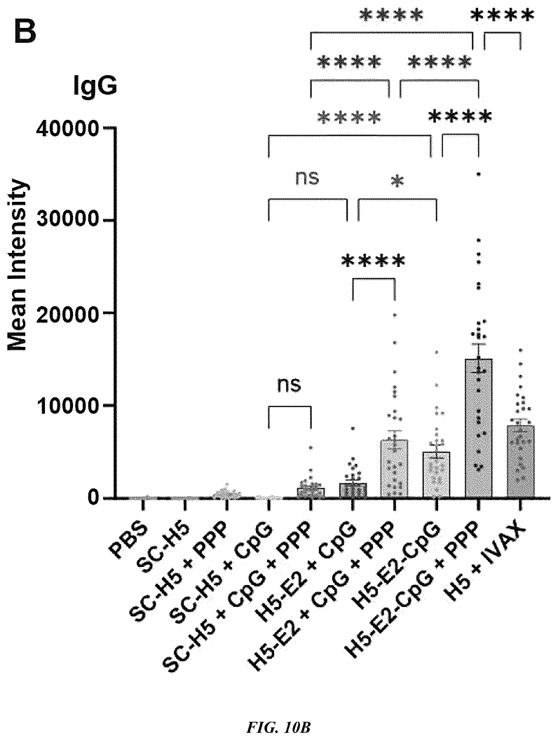

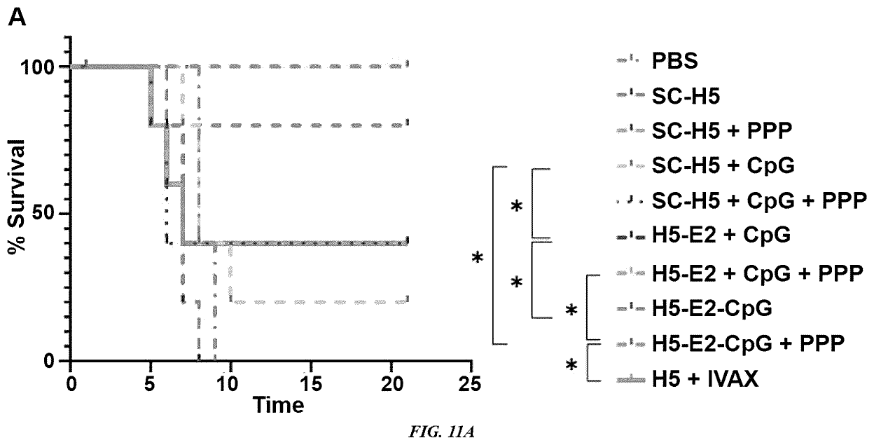

Antibody response elicited by H5-CpG-E2 loaded hydrogel vaccine against 28 different H5 variants. It was found that PPP promoted an increase the in homosubtypic cross-reactivity (e.g., see FIG. 10A). Further, PPP promoted the increase in the antibody level against 28 homosubtypic variants of the vaccinated variant when H5 is conjugated on E2. (e.g., see FIG. 10B). The H5-CpG-E2/PPP vaccine exhibited a significant higher IgG against the 28 homosubtypic variants than the H5-IVAX single dose group (used as a positive control in this study). Conjugating both H5 and CpG on E2 helps to increase the homosubtypic cross-reactivity no matter if loaded in PPP or in bolus shot.

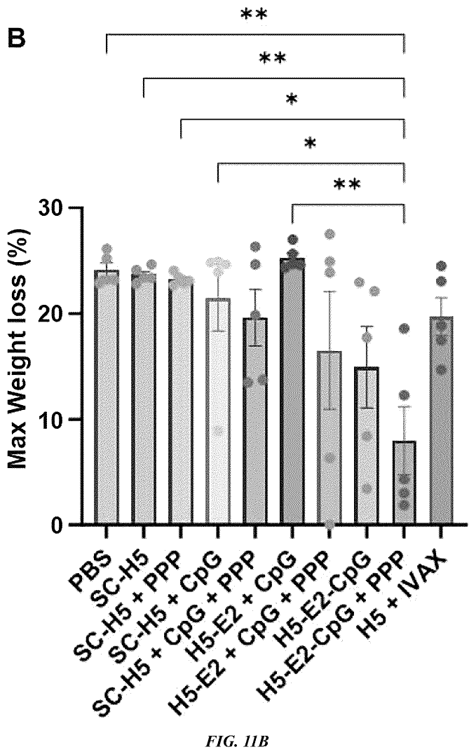

Immunization with H5-E2-CpG loaded hydrogel protects mice from the lethal challenge of influenza and improves morbidity. It was found that H5-E2-CpG/PPP group had the highest survival rate. All mice from that group survived from the H5N1 challenge, and this was the only group to have 100% survival (e.g., see FIG. 11A). The H5-E2-CPG/PPP group had the lowest average weight loss. Even though the difference between H5-E2-CpG with and without PPP hydrogel groups are not significant, the average weight loss of H5-E2-CpG/PPP group is about half of that of the H5-E2-CpG group and 40% of the H5+IVAX group (e.g., see FIG. 11B).

A number of embodiments have been described herein. Nevertheless, it will be understood that various modifications may be made without departing from the spirit and scope of this disclosure. Accordingly, other embodiments are within the scope of the following claims.

What is claimed is:

1. A vaccine depot formulation for the simultaneous delivery of an antigen and an adjuvant, comprising:
a biodegradable thermosensitive hydrogel that has been loaded or embedded with nanoparticles that comprise the antigen and the adjuvant,
wherein the nanoparticles are hollow and comprise an outer surface and an accessible inner cavity, where the antigen and/or the adjuvant is conjugated to the outer surface of the nanoparticles, and/or where the antigen and/or the adjuvant is conjugated or loaded into the inner cavity of the nanoparticles.

2. The vaccine depot formulation of claim 1, wherein the vaccine depot formulation is suitable for injection by the biodegradable thermosensitive hydrogel being in a liquid state at normal ambient temperatures but solidifying into a gel like state at a normal body temperature.

3. The vaccine depot formulation of claim 1, wherein the biodegradable thermosensitive hydrogel is selected from poloxamer-407/188, poloxamer-407, chitosan, poloxamer-chitosan, cellulose, methylcellulose, sodium carboxymethyl cellulose, PLGA-PEG-PLGA (PPP), PCL-PEG-PCL, and poly(N-isopropylacrylamide).

4. The vaccine depot formulation of claim 1, wherein the biodegradable thermosensitive hydrogel is PPP.

5. The vaccine depot formulation of claim 1, wherein the antigen is a foreign antigen from a pathogen selected from a bacterium, a fungus or a virus.

6. The vaccine depot formulation of claim 5, wherein the bacterium is selected from Actinomyces israelii, Bacillus anthracis, Bacillus cereus, Bartonella henselae, Bartonella quintana, Bordetella pertussis, Borrelia burgdorferi, Borrelia garinii, Borrelia afzelii, Borrelia recurrentis, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diphtheriae, Enterococcus faecalis, Enterococcus faecium, Escherichia coli, Francisella tularensis, Haemophilus influenzae, Helicobacter pylori, Legionella pneumophila, Leptospira interrogans, Leptospira santarosai, Leptospira weilii, Leptospira noguchii, Listeria monocytogenes, Mycobacterium leprae, Mycobacterium tuberculosis, Mycobacterium ulcerans, Mycoplasma pneumoniae, Neisseria gonorrhoeae, Neisseria meningitidis, Pseudomonas aeruginosa, Rickettsia rickettsia, Salmonella typhi, Salmonella typhimurium, Shigella sonnei, Staphylococcus aureus, Staphylococcus epidermidis, Staphylococcus saprophyticus, Streptococcus agalactiae, Streptococcus pneumoniae, Streptococcus pyogenes, Treponema pallidum, Ureaplasma urealyticum, Vibrio cholerae, Yersinia pestis, Yersinia enterocolitica, and Yersinia pseudotuberculosis.

7. The vaccine depot formulation of claim 5, wherein the fungus is selected from Absidia corymbifera, Absidia ramose, Achorion gallinae, Actinomadura spp., Ajellomyces dermatididis, Aleurisma brasiliensis, Allersheria boydii, Arthroderma spp., Aspergillus flavus, Aspergillus fumigatu, Basidiobolus spp, Blastomyces spp, Cadophora spp, Candida albicans, Cercospora apii, Chrysosporium spp, Cladosporium spp, Cladothrix asteroids, Coccidioides immitis, Cryptococcus albidus, Cryptococcus gattii, Cryptococcus laurentii, Cryptococcus neoformans, Cunninghamella elegans, Dematium wernecke, Discomyces israelii, Emmonsia spp, Emmonsiella capsulate, Endomyces geotrichum, Entomophthora coronate, Epidermophyton floccosum, Filobasidiella neoformans, Fonsecaea spp., Geotrichum candidum, Glenospora khartoumensis, Gymnoascus gypseus, Haplosporangium parvum, Histoplasma, Histoplasma capsulatum, Hormiscium dermatididis, Hormodendrum spp., Keratinomyces spp, Langeronia soudanense, Leptosphaeria senegalensis, Lichtheimia corymbifera, Lobmyces loboi, Loboa loboi, Lobomycosis, Madurella spp., Malassezia furfur, Micrococcus pelletieri, Microsporum spp, Monilia spp., Mucor spp., Mycobacterium tuberculosis, Nannizzia spp., Neotestudina rosatii, Nocardia spp., Oidium albicans, Oospora lactis, Paracoccidioides brasiliensis, Petriellidium boydii, Phialophora spp., Piedraia hortae, Pityrosporum furfur, Pneumocystis jirovecii (or Pneumocystis carinii), Pullularia gougerotii, Pyrenochaeta romeroi, Rhinosporidium seeberi, Sabouraudites (Microsporum), Sartorya fumigate, Sepedonium, Sporotrichum spp., Stachybotrys, Stachybotrys chartarum, Streptomyce spp., Tinea spp., Torula spp, Trichophyton spp, Trichosporon spp, and Zopfia rosatii.

8. The vaccine depot formulation of claim 5, wherein the virus is selected from Human coronavirus, Human papillomavirus, Torque teno virus, Barmah forest virus, Chikungunya virus, Eastern equine encephalitis virus, Mayaro virus, O'nyong-nyong virus, Ross river virus, Sagiyama virus, Semliki forest virus, Sindbis virus, Venezuelan equine encephalitis virus, Western equine encephalitis virus, Junin arenavirus, Lassa virus, Lymphocytic choriomeningitis virus, Machupo virus, Pichinde virus, Human SARS coronavirus, MERS coronavirus, SARS coronavirus, Encephalomyocarditis virus, Cosavirus A, Human cytomegalovirus, Human T-lymphotropic virus, Hepatitis delta virus, Adeno-associated virus, Ebolavirus, Human rhinovirus, Coxsackievirus, Echovirus, Human enterovirus, Poliovirus, Human parvovirus B19, Murray valley encephalitis virus, Dengue virus, Japanese encephalitis virus, Langat virus, Louping ill virus, St. louis encephalitis virus, Tick-borne powassan virus, West Nile virus, Yellow fever virus, Zika virus, Hantaan virus, New York virus, Puumala virus, Seoul virus, Hendra virus, Nipah virus, Hepatitis virus, Influenza virus, Aichi virus, Human immunodeficiency virus, Cercopithecine herpesvirus, Epstein-Barr virus, Australian bat lyssavirus, Duvenhage virus, Lagos bat virus, Mokola virus, Rabies virus, European bat lyssavirus, Human astrovirus, Lake Victoria marburgvirus, Human adenovirus, Molluscum contagiosum virus, Measles virus, Human papillomavirus, Crimean-Congo hemorrhagic fever virus, Dugbe virus, Norwalk virus, Southampton virus, Oropouche virus, Bunyamwera virus, Bunyavirus La Crosse, Bunyavirus snowshoe hare, Hepatitis B virus, Human respiratory syncytial virus, Monkeypox virus, Cowpox virus, Horsepox virus, Vaccinia virus, Variola virus, Yaba monkey tumor virus, Yaba-like disease virus, Orf virus, GB virus C/Hepatitis G virus, Punta toro phlebovirus, Rift valley fever virus, Sandfly fever Naples phlebovirus (Toscana virus), Sandfly fever sicilian virus, Uukuniemi virus, BK polyomavirus, JC polyomavirus, KI Polyomavirus, Merkel cell polyomavirus, WU polyomavirus, Human parainfluenza, Rosavirus, Human herpesvirus, Rotavirus, Rubella virus, Mammalian orthorubulavirus (Simian virus), Mumps virus, Salivirus A, Sapporo virus, Banna virus, Eastern chimpanzee simian foamy virus, Simian foamy virus, Dhori virus, Vientovirus, Human torovirus, Varicella-zoster virus, Chandipura virus, Isfahan virus, and Vesicular stomatitis virus.

9. The vaccine depot formulation of claim 5, wherein the antigen is from an influenza virus, SARS-CoV-2, or Coxiella burnetii.

10. The vaccine depot formulation of claim 1, wherein the antigen is an endogenous antigen from a cancer or is associated with an immune disease.

11. The vaccine depot formulation of claim 10, wherein the antigen is a cancer tumor-specific antigen selected from NY-ESO-1, gp100, CT26, alphafetoprotein (AFP), carcinoembryonic antigen (CEA), CA-125, CA15-3, CA19-9, MUC-1, epithelial tumor antigen (ETA), tyrosinase, melanoma-associated antigen (MAGE), abnormal products of ras or p53, CTAG1B, MAGEA1, and HER2/neu.

12. The vaccine depot formulation of claim 1, wherein the nanoparticles are hollow protein nanoparticles.

13. The vaccine depot formulation of claim 12, wherein the hollow protein nanoparticles are based on the E2 subunit, or a portion thereof, of the pyruvate dehydrogenase complex (PDC) from the Geobacillus stearothermophilus.

14. The vaccine depot formulation of claim 13, wherein the E2 subunit of the PDC has been recombinantly modified to substitute one or more amino acids with cysteines.

15. The vaccine depot formulation of claim 1, wherein the adjuvant is selected from CpG1826, CpG1018, MGN1703, ssRNA, aluminum hydroxide, aluminum phosphate, and aluminum potassium sulfate, AS04, MF59, AS01_{B}, CpG ODN 1466, CpG ODN PB3, CpG ODN BW005, CpG Alum, CPG 21424, CpG 7909, CpG-ODN 2135, ODN K3, CpG ODN 10101, CpG-28, CpG ODN C274, CpG ODN C695, CpG ODN #17, CpG-ODN 2722, CpG 8916, CpG 8954, CpG ODN 678, CpG ODN BW015, CpG ODN 658, CpG ODN 640, CpG ODN PB9, CpG ODN BW004, CpG ODN BW103, CpG ODN 110, CpG ODN BW206, CpG ODN 607, CpG ODN 647, CpG ODN 111, CpG ODN 109, CpG ODN 656, CpG ODN 664, CpG ODN C9, CPG2429, CPG5475, CPG21608, CPG21797, CPG21796, CPG21799, CPG21800, CPG21802, CPG21889, CPG23409, CpG-c41, CPG23410, CPG23411, CPG23412, CPG23413, CPG23617, CPG23414, CPG 1681, CPG 2143, CPG 21425, and CPG 21426.

16. The vaccine depot formulation of claim 15, wherein the adjuvant is CpG1018 or CpG1826.

17. A method of immunizing a subject comprising:
administering one or more doses of the vaccine depot formulation of claim 1 to protect a subject from an infection by a pathogen and/or from a disease.

18. The method of claim 17, wherein the infection and/or disease is selected from Q fever, avian influenza, and SARS-CoV-2.

19. The method of claim 17, wherein the vaccine depot formulation is formulated for intramuscular administration, subcutaneous administration, or intravenous administration.

20. The method of claim 17, wherein the vaccine depot formulation provides long-lasting, sustained delivery of the antigen for at least 4 or more weeks in vivo.


SEQUENCE LISTING (45 sequences)

SEQ ID NO: 1 - protein, 428 aa, Geobacillus stearothermophilus
MAFEFKLPDIGEGIHEGEIVKWFVKPGDEVNEDDVLCEVQNDKAVVEIPSPVKGKVLEILVPEGTVATVGQTLITLDAPGYENMTFKGQEQEEAKKEEKTETVSKEEKVDAVAPNAPAAEAEAGPNRRVIAMPSVRKYAREKGVDIRLVQGTGKNGRVLKEDIDAFLAGGAKPAPAAAEEKAAPAAAKPATTEGEFPETREKMSGIRRAIAKAMVHSKHTAPHVTLMDEADVTKLVAHRKKFKAIAAEKGIKLTFLPYVVKALVSALREYPVLNTSIDDETEEIIQKHYYNIGIAADTDRGLLVPVIKHADRKPIFALAQEINELAEKARDGKLTPGEMKGASCTITNIGSAGGQWFTPVINHPEVAILGIGRIAEKPIVRDGEIVAAPMLALSLSFDHRMIDGATAQKALNHIKRLLSDPELLLMEA

SEQ ID NO: 2 - DNA, 783 bp, synthetic construct
  source 1..783: mol_type = other DNA
atgctgtctgttcctggtcccgctgctgcagaggaaaaggctgctccagcggctgcgaaaccggctactactgaaggtgaattccctgaaacccgtgaaaaaatgtctggtatccgtcgtgcaatcgcgaaagccatggttcactctaaacacaccgcgccacacgttaccctgatggatgaagcagacgttaccaaactggttgcgcaccgtaaaaaattcaaggcgattgcggcggaaaaaggtatcaaactgaccttcctgccgtacgttgttaaagctctggtttcggctctgcgtgaatacccggttctgaacacctctattgacgactgcaccgaagaaatcatccagaaacactactacaacatcggtatcgctgcggacactgatcgtggtctgctggttcctgtgattaaacacgcggaccgtaaaccgatcttcgcgctcgctcaggaaatcaacgaactggctgagaaagctcgtgacggtaaactgactcctggtgaaatgaaaggcgcgtcttgcactattaccaacatcggctctgcaggtggtcagtggttcaccccagttatcaaccacccggaagttgcgatcctgggtattggtcgtatagccgaaaagccgatcgttcgtgacggtgaaatcgttgctgctccgatgctggccctgtctctgtctttcgatcatcgtatgattgatggcgcgaccgcacagaaagccctgaaccacatcaaacgtctgctgtccgacccggaactgctgctgatggaagcttaa

SEQ ID NO: 3 - protein, 260 aa, synthetic construct
MLSVPGPAAAEEKAAPAAAKPATTEGEFPETREKMSGIRRAIAKAMVHSKHTAPHVTLMDEADVTKLVAHRKKFKAIAAEKGIKLTFLPYVVKALVSALREYPVLNTSIDDCTEEIIQKHYYNIGIAADTDRGLLVPVIKHADRKPIFALAQEINELAEKARDGKLTPGEMKGASCTITNIGSAGGQWFTPVINHPEVAILGIGRIAEKPIVRDGEIVAAPMLALSLSFDHRMIDGATAQKALNHIKRLLSDPELLLMEA

SEQ ID NO: 4 - DNA, 783 bp, synthetic construct
  source 1..783: mol_type = other DNA
atgctgtctgttcctggtcccgctgctgcagaggaaaaggctgctccagcggctgcgaaaccggctactactgaaggtgaattccctgaaacccgtgaaaaaatgtctggtatccgtcgtgcaatcgcgaaagccatggttcactctaaacacaccgcgccacacgttaccctgatggatgaagcagacgttaccaaactggttgcgcaccgtaaaaaattcaaggcgattgcggcggaaaaaggtatcaaactgaccttcctgccgtacgttgttaaagctctggtttcggctctgcgtgaatacccggttctgaacacctctattgacgacgagaccgaagaaatcatccagaaacactactacaacatcggtatcgctgcggacactgatcgtggtctgctggttcctgtgattaaacacgcggaccgtaaaccgatcttcgcgctcgctcaggaaatcaacgaactggctgagaaagctcgtgacggtaaactgactcctggtgaaatgaaaggcgcgtcttgcactattaccaacatcggctctgcaggtggtcagtggttcaccccagttatcaaccacccggaagttgcgatcctgggtattggtcgtatagccgaaaagccgatcgttcgttgcggtgaaatcgttgctgctccgatgctggccctgtctctgtctttcgatcatcgtatgattgatggcgcgaccgcacagaaagccctgaaccacatcaaacgtctgctgtccgacccggaactgctgctgatggaagcttaa

SEQ ID NO: 5 - protein, 260 aa, synthetic construct
MLSVPGPAAAEEKAAPAAAKPATTEGEFPETREKMSGIRRAIAKAMVHSKHTAPHVTLMDEADVTKLVAHRKKFKAIAAEKGIKLTFLPYVVKALVSALREYPVLNTSIDDETEEIIQKHYYNIGIAADTDRGLLVPVIKHADRKPIFALAQEINELAEKARDGKLTPGEMKGASCTITNIGSAGGQWFTPVINHPEVAILGIGRIAEKPIVRCGEIVAAPMLALSLSFDHRMIDGATAQKALNHIKRLLSDPELLLMEA

SEQ ID NO: 6 - DNA, 840 bp, synthetic construct
  source 1..840: mol_type = other DNA
gctagcaccggcaaaaatggtcgtgtgctgaaagaagacattgatgcgtttctggcgggcggcgcgaaacccgggcccgctgctgcagaggaaaaggctgctccagcggctgcgaaaccggctactactgaaggtgaattccctgaaacccgtgaaaaaatgtctggtatccgtcgtgcaatcgcgaaagccatggttcactctaaacacaccgcgccacacgttaccctgatggatgaagcagacgttaccaaactggttgcgcaccgtaaaaaattcaaggcgattgcggcggaaaaaggtatcaaactgaccttcctgccgtacgttgttaaagctctggtttcggctctgcgtgaatacccggttctgaacacctctattgacgacgagaccgaagaaatcatccagaaacactactacaacatcggtatcgctgcggacactgatcgtggtctgctggttcctgtgattaaacacgcggaccgtaaaccgatcttcgcgctcgctcaggaaatcaacgaactggctgagaaagctcgtgacggtaaactgactcctggtgaaatgaaaggcgcgtcttgcactattaccaacatcggctctgcaggtggtcagtggttcaccccagttatcaaccacccggaagttgcgatcctgggtattggtcgtatagccgaaaagccgatcgttcgttgcggtgaaatcgttgctgctccgatgctggccctgtctctgtctttcgatcatcgtatgattgatggcgcgaccgcacagaaagccctgaaccacatcaaacgtctgctgtccgacccggaactgctgctgatggaagcttaa

SEQ ID NO: 7 - protein, 279 aa, synthetic construct
ASTGKNGRVLKEDIDAFLAGGAKPGPAAAEEKAAPAAAKPATTEGEFPETREKMSGIRRAIAKAMVHSKHTAPHVTLMDEADVTKLVAHRKKFKAIAAEKGIKLTFLPYVVKALVSALREYPVLNTSIDDETEEIIQKHYYNIGIAADTDRGLLVPVIKHADRKPIFALAQEINELAEKARDGKLTPGEMKGASCTITNIGSAGGQWFTPVINHPEVAILGIGRIAEKPIVRCGEIVAAPMLALSLSFDHRMIDGATAQKALNHIKRLLSDPELLLMEA

SEQ ID NO: 8 - DNA, 822 bp, synthetic construct
  source 1..822: mol_type = other DNA
gctagcgtgctgaaagaagacattgatgcgtttctggcgggcggcgcgaaacccgggcccgctgctgcagaggaaaaggctgctccagcggctgcgaaaccggctactactgaaggtgaattccctgaaacccgtgaaaaaatgtctggtatccgtcgtgcaatcgcgaaagccatggttcactctaaacacaccgcgccacacgttaccctgatggatgaagcagacgttaccaaactggttgcgcaccgtaaaaaattcaaggcgattgcggcggaaaaaggtatcaaactgaccttcctgccgtacgttgttaaagctctggtttcggctctgcgtgaatacccggttctgaacacctctattgacgacgagaccgaagaaatcatccagaaacactactacaacatcggtatcgctgcggacactgatcgtggtctgctggttcctgtgattaaacacgcggaccgtaaaccgatcttcgcgctcgctcaggaaatcaacgaactggctgagaaagctcgtgacggtaaactgactcctggtgaaatgaaaggcgcgtcttgcactattaccaacatcggctctgcaggtggtcagtggttcaccccagttatcaaccacccggaagttgcgatcctgggtattggtcgtatagccgaaaagccgatcgttcgttgcggtgaaatcgttgctgctccgatgctggccctgtctctgtctttcgatcatcgtatgattgatggcgcgaccgcacagaaagccctgaaccacatcaaacgtctgctgtccgacccggaactgctgctgatggaagcttaa

SEQ ID NO: 9 - protein, 273 aa, synthetic construct
ASVLKEDIDAFLAGGAKPGPAAAEEKAAPAAAKPATTEGEFPETREKMSGIRRAIAKAMVHSKHTAPHVTLMDEADVTKLVAHRKKFKAIAAEKGIKLTFLPYVVKALVSALREYPVLNTSIDDETEEIIQKHYYNIGIAADTDRGLLVPVIKHADRKPIFALAQEINELAEKARDGKLTPGEMKGASCTITNIGSAGGQWFTPVINHPEVAILGIGRIAEKPIVRCGEIVAAPMLALSLSFDHRMIDGATAQKALNHIKRLLSDPELLLMEA

SEQ ID NO: 10 - DNA, 672 bp, Coxiella burnetii
  source 1..672: mol_type = genomic DNA
ccgcagcaagtcaaagacattcagtcaatcgttcatcattacctggtcaatcacccggaagtcctggttgaagccagccaggcactgcagaagaaaaccgaagctcagcaagaagaacatgctcagcaagcgatcaaagaaaacgcgaaaaaactgttcaacgatccggcatcaccggtcgcaggtaacccgcatggtaatgtcaccctggtggaatttttcgactatcagtgcggccactgtaaagctatgaattcggtgattcaggcgatcgttaaacagaacaaaaatctgcgtgtggtttttaaagaactgccgattttcggcggtcagagtcaatacgcggccaaagtctccctggcagctgcgaaacagggcaaatattacgcttttcacgatgcgctgctgagcgtggacggtcaactgtctgaacagattaccctgcaaacggccgaaaaagttggcctgaacgtcgcacagctgaaaaaagatatggacaatccggccatccagaaacaactgcgcgataacttccagctggcccaaagtctgcagctggcaggcaccccgacgtttgtgattggtaacaaagcgctgaccaaattcggtttcattccgggcgcaacgtcgcaacaaaacctgcaaaaagaaattgaccgtgtcgaaaaataa

SEQ ID NO: 11 - protein, 225 aa, Coxiella burnetii
PQQVKDIQSIVHHYLVNHPEVLVEASQALQKKTEAQQEEHAQQAIKENAKKLFNDPASPVAGNPHGNVTLVEFFDYQCGHCKAMNSVIQAIVKQNKNLRVVFKELPIFGGQSQYAAKVSLAAAKQGKYYAFHDALLSVDGQLSEQITLQTAEKVGLNVAQLKKDMDNPAIQKQLRDNFQLAQSLQLAGTPTFVIGNKALTKFGFIPGATSQQNLQKEIDRVEKST

SEQ ID NO: 12 - DNA, 1509 bp, synthetic construct
  source 1..1509: mol_type = other DNA
ccgcagcaagtcaaagacattcagtcaatcgttcatcattacctggtcaatcacccggaagtcctggttgaagccagccaggcactgcagaagaaaaccgaagctcagcaagaagaacatgctcagcaagcgatcaaagaaaacgcgaaaaaactgttcaacgatccggcatcaccggtcgcaggtaacccgcatggtaatgtcaccctggtggaatttttcgactatcagtgcggccactgtaaagctatgaattcggtgattcaggcgatcgttaaacagaacaaaaatctgcgtgtggtttttaaagaactgccgattttcggcggtcagagtcaatacgcggccaaagtctccctggcagctgcgaaacagggcaaatattacgcttttcacgatgcgctgctgagcgtggacggtcaactgtctgaacagattaccctgcaaacggccgaaaaagttggcctgaacgtcgcacagctgaaaaaagatatggacaatccggccatccagaaacaactgcgcgataacttccagctggcccaaagtctgcagctggcaggcaccccgacgtttgtgattggtaacaaagcgctgaccaaattcggtttcattccgggcgcaacgtcgcaacaaaacctgcaaaaagaaattgaccgtgtcgaaaaagctagcaccggcaaaaatggtcgtgtgctgaaagaagacattgatgcgtttctggcgggcggcgcgaaacccgggcccgctgctgcagaggaaaaggctgctccagcggctgcgaaaccggctactactgaaggtgaattccctgaaacccgtgaaaaaatgtctggtatccgtcgtgcaatcgcgaaagccatggttcactctaaacacaccgcgccacacgttaccctgatggatgaagcagacgttaccaaactggttgcgcaccgtaaaaaattcaaggcgattgcggcggaaaaaggtatcaaactgaccttcctgccgtacgttgttaaagctctggtttcggctctgcgtgaatacccggttctgaacacctctattgacgacgagaccgaagaaatcatccagaaacactactacaacatcggtatcgctgcggacactgatcgtggtctgctggttcctgtgattaaacacgcggaccgtaaaccgatcttcgcgctcgctcaggaaatcaacgaactggctgagaaagctcgtgacggtaaactgactcctggtgaaatgaaaggcgcgtcttgcactattaccaacatcggctctgcaggtggtcagtggttcaccccagttatcaaccacccggaagttgcgatcctgggtattggtcgtatagccgaaaagccgatcgttcgttgcggtgaaatcgttgctgctccgatgctggccctgtctctgtctttcgatcatcgtatgattgatggcgcgaccgcacagaaagccctgaaccacatcaaacgtctgctgtccgacccggaactgctgctgatggaagcttaa

SEQ ID NO: 13 - protein, 502 aa, synthetic construct
PQQVKDIQSIVHHYLVNHPEVLVEASQALQKKTEAQQEEHAQQAIKENAKKLFNDPASPVAGNPHGNVTLVEFFDYQCGHCKAMNSVIQAIVKQNKNLRVVFKELPIFGGQSQYAAKVSLAAAKQGKYYAFHDALLSVDGQLSEQITLQTAEKVGLNVAQLKKDMDNPAIQKQLRDNFQLAQSLQLAGTPTFVIGNKALTKFGFIPGATSQQNLQKEIDRVEKASTGKNGRVLKEDIDAFLAGGAKPGPAAAEEKAAPAAAKPATTEGEFPETREKMSGIRRAIAKAMVHSKHTAPHVTLMDEADVTKLVAHRKKFKAIAAEKGIKLTFLPYVVKALVSALREYPVLNTSIDDETEEIIQKHYYNIGIAADTDRGLLVPVIKHADRKPIFALAQEINELAEKARDGKLTPGEMKGASCTITNIGSAGGQWFTPVINHPEVAILGIGRIAEKPIVRCGEIVAAPMLALSLSFDHRMIDGATAQKALNHIKRLLSDPELLLMEA

SEQ ID NO: 14 - DNA, 1491 bp, synthetic construct
  source 1..1491: mol_type = other DNA
ccgcagcaagtcaaagacattcagtcaatcgttcatcattacctggtcaatcacccggaagtcctggttgaagccagccaggcactgcagaagaaaaccgaagctcagcaagaagaacatgctcagcaagcgatcaaagaaaacgcgaaaaaactgttcaacgatccggcatcaccggtcgcaggtaacccgcatggtaatgtcaccctggtggaatttttcgactatcagtgcggccactgtaaagctatgaattcggtgattcaggcgatcgttaaacagaacaaaaatctgcgtgtggtttttaaagaactgccgattttcggcggtcagagtcaatacgcggccaaagtctccctggcagctgcgaaacagggcaaatattacgcttttcacgatgcgctgctgagcgtggacggtcaactgtctgaacagattaccctgcaaacggccgaaaaagttggcctgaacgtcgcacagctgaaaaaagatatggacaatccggccatccagaaacaactgcgcgataacttccagctggcccaaagtctgcagctggcaggcaccccgacgtttgtgattggtaacaaagcgctgaccaaattcggtttcattccgggcgcaacgtcgcaacaaaacctgcaaaaagaaattgaccgtgtcgaaaaagctagcgtgctgaaagaagacattgatgcgtttctggcgggcggcgcgaaacccgggcccgctgctgcagaggaaaaggctgctccagcggctgcgaaaccggctactactgaaggtgaattccctgaaacccgtgaaaaaatgtctggtatccgtcgtgcaatcgcgaaagccatggttcactctaaacacaccgcgccacacgttaccctgatggatgaagcagacgttaccaaactggttgcgcaccgtaaaaaattcaaggcgattgcggcggaaaaaggtatcaaactgaccttcctgccgtacgttgttaaagctctggtttcggctctgcgtgaatacccggttctgaacacctctattgacgacgagaccgaagaaatcatccagaaacactactacaacatcggtatcgctgcggacactgatcgtggtctgctggttcctgtgattaaacacgcggaccgtaaaccgatcttcgcgctcgctcaggaaatcaacgaactggctgagaaagctcgtgacggtaaactgactcctggtgaaatgaaaggcgcgtcttgcactattaccaacatcggctctgcaggtggtcagtggttcaccccagttatcaaccacccggaagttgcgatcctgggtattggtcgtatagccgaaaagccgatcgttcgttgcggtgaaatcgttgctgctccgatgctggccctgtctctgtctttcgatcatcgtatgattgatggcgcgaccgcacagaaagccctgaaccacatcaaacgtctgctgtccgacccggaactgctgctgatggaagcttaa

SEQ ID NO: 15 - protein, 496 aa, synthetic construct
PQQVKDIQSIVHHYLVNHPEVLVEASQALQKKTEAQQEEHAQQAIKENAKKLFNDPASPVAGNPHGNVTLVEFFDYQCGHCKAMNSVIQAIVKQNKNLRVVFKELPIFGGQSQYAAKVSLAAAKQGKYYAFHDALLSVDGQLSEQITLQTAEKVGLNVAQLKKDMDNPAIQKQLRDNFQLAQSLQLAGTPTFVIGNKALTKFGFIPGATSQQNLQKEIDRVEKASVLKEDIDAFLAGGAKPGPAAAEEKAAPAAAKPATTEGEFPETREKMSGIRRAIAKAMVHSKHTAPHVTLMDEADVTKLVAHRKKFKAIAAEKGIKLTFLPYVVKALVSALREYPVLNTSIDDETEEIIQKHYYNIGIAADTDRGLLVPVIKHADRKPIFALAQEINELAEKARDGKLTPGEMKGASCTITNIGSAGGQWFTPVINHPEVAILGIGRIAEKPIVRCGEIVAAPMLALSLSFDHRMIDGATAQKALNHIKRLLSDPELLLMEA

SEQ ID NO: 16 - DNA, 39 bp, synthetic construct
  source 1..39: mol_type = other DNA
gcccacatcgttatggtggatgcctacaagccaactaaa

SEQ ID NO: 17 - protein, 13 aa, synthetic construct
AHIVMVDAYKPTK

SEQ ID NO: 18 - DNA, 417 bp, synthetic construct
  source 1..417: mol_type = other DNA
atgtcgtactaccatcaccatcaccatcacgattacgacatcccaacgaccgaaaacctgtattttcagggcgccatggttgataccttatcaggtttatcaagtgagcaaggtcagtccggtgatatgacaattgaagaagatagtgctacccatattaaattctcaaaacgtgatgaggacggcaaagagttagctggtgcaactatggagttgcgtgattcatctggtaaaactattagtacatggatttcagatggacaagtgaaagatttctacctgtatccaggaaaatatacatttgtcgaaaccgcagcaccagacggttatgaggtagcaactgctattacctttacagttaatgagcaaggtcaggttactgtaaatggcaaagcaactaaaggtgacgctcatatt

SEQ ID NO: 19 - protein, 139 aa, synthetic construct
MSYYHHHHHHDYDIPTTENLYFQGAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHI

SEQ ID NO: 20 - DNA, 855 bp, synthetic construct
  source 1..855: mol_type = other DNA
atggcccacatcgttatggtggatgcctacaagccaactaaaggttcaggaacagcaggtggtgggtcaggttccctgtctgttcctggtcccgctgctgcagaggaaaaggctgctccagcggctgcgaaaccggctactactgaaggtgaattccctgaaacccgtgaaaaaatgtctggtatccgtcgtgcaatcgcgaaagccatggttcactctaaacacaccgcgccacacgttaccctgatggatgaagcagacgttaccaaactggttgcgcaccgtaaaaaattcaaggcgattgcggcggaaaaaggtatcaaactgaccttcctgccgtacgttgttaaagctctggtttcggctctgcgtgaatacccggttctgaacacctctattgacgacgagaccgaagaaatcatccagaaacactactacaacatcggtatcgctgcggacactgatcgtggtctgctggttcctgtgattaaacacgcggaccgtaaaccgatcttcgcgctcgctcaggaaatcaacgaactggctgagaaagctcgtgacggtaaactgactcctggtgaaatgaaaggcgcgtcttgcactattaccaacatcggctctgcaggtggtcagtggttcaccccagttatcaaccacccggaagttgcgatcctgggtattggtcgtatagccgaaaagccgatcgttcgttgcggtgaaatcgttgctgctccgatgctggccctgtctctgtctttcgatcatcgtatgattgatggcgcgaccgcacagaaagccctgaaccacatcaaacgtctgctgtccgacccggaactgctgctgatggaagcttaa

SEQ ID NO: 21 - protein, 284 aa, synthetic construct
MAHIVMVDAYKPTKGSGTAGGGSGSLSVPGPAAAEEKAAPAAAKPATTEGEFPETREKMSGIRRAIAKAMVHSKHTAPHVTLMDEADVTKLVAHRKKFKAIAAEKGIKLTFLPYVVKALVSALREYPVLNTSIDDETEEIIQKHYYNIGIAADTDRGLLVPVIKHADRKPIFALAQEINELAEKARDGKLTPGEMKGASCTITNIGSAGGQWFTPVINHPEVAILGIGRIAEKPIVRCGEIVAAPMLALSLSFDHRMIDGATAQKALNHIKRLLSDPELLLMEA

SEQ ID NO: 22 - DNA, 1128 bp, synthetic construct
  source 1..1128: mol_type = other DNA
atgtcgtactaccatcaccatcaccatcacgattacgacatcccaacgaccgaaaacctgtattttcagggcgccatggttgataccttatcaggtttatcaagtgagcaaggtcagtccggtgatatgacaattgaagaagatagtgctacccatattaaattctcaaaacgtgatgaggacggcaaagagttagctggtgcaactatggagttgcgtgattcatctggtaaaactattagtacatggatttcagatggacaagtgaaagatttctacctgtatccaggaaaatatacatttgtcgaaaccgcagcaccagacggttatgaggtagcaactgctattacctttacagttaatgagcaaggtcaggttactgtaaatggcaaagcaactaaaggtgacgctcatattgctagcggttcaggaacagcaggtggtgggtcaggttccccgcagcaagtcaaagacattcagtcaatcgttcatcattacctggtcaatcacccggaagtcctggttgaagccagccaggcactgcagaagaaaaccgaagctcagcaagaagaacatgctcagcaagcgatcaaagaaaacgcgaaaaaactgttcaacgatccggcatcaccggtcgcaggtaacccgcatggtaatgtcaccctggtggaatttttcgactatcagtgcggccactgtaaagctatgaattcggtgattcaggcgatcgttaaacagaacaaaaatctgcgtgtggtttttaaagaactgccgattttcggcggtcagagtcaatacgcggccaaagtctccctggcagctgcgaaacagggcaaatattacgcttttcacgatgcgctgctgagcgtggacggtcaactgtctgaacagattaccctgcaaacggccgaaaaagttggcctgaacgtcgcacagctgaaaaaagatatggacaatccggccatccagaaacaactgcgcgataacttccagctggcccaaagtctgcagctggcaggcaccccgacgtttgtgattggtaacaaagcgctgaccaaattcggtttcattccgggcgcaacgtcgcaacaaaacctgcaaaaagaaattgaccgtgtcgaaaaataa

SEQ ID NO: 23 - protein, 375 aa, synthetic construct
MSYYHHHHHHDYDIPTTENLYFQGAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHIASGSGTAGGGSGSPQQVKDIQSIVHHYLVNHPEVLVEASQALQKKTEAQQEEHAQQAIKENAKKLFNDPASPVAGNPHGNVTLVEFFDYQCGHCKAMNSVIQAIVKQNKNLRVVFKELPIFGGQSQYAAKVSLAAAKQGKYYAFHDALLSVDGQLSEQITLQTAEKVGLNVAQLKKDMDNPAIQKQLRDNFQLAQSLQLAGTPTFVIGNKALTKFGFIPGATSQQNLQKEIDRVEK

SEQ ID NO: 24 - DNA, 30 bp, synthetic construct
  source 1..30: mol_type = other DNA
gccgatcgttcgttgcggtgaaatcgttgc

SEQ ID NO: 25 - DNA, 26 bp, synthetic construct
  source 1..26: mol_type = other DNA
ttttcggctatacgaccaatacccag

SEQ ID NO: 26 - DNA, 48 bp, synthetic construct
  source 1..48: mol_type = other DNA
catatgcaccatcaccatcaccatccgcagcaagtcaaagacattcag

SEQ ID NO: 27 - DNA, 24 bp, synthetic construct
  source 1..24: mol_type = other DNA
gctagcttagccgccggtttccgg

SEQ ID NO: 28 - protein, 15 aa, synthetic construct
HYLVNHPEVLVEASQ

SEQ ID NO: 29 - DNA, 30 bp, synthetic construct; identical to SEQ ID NO: 24
  source 1..30: mol_type = other DNA

SEQ ID NO: 30 - DNA, 26 bp, synthetic construct
  source 1..26: mol_type = other DNA
ttttcggctatacgaccaatacccag

SEQ ID NO: 31 - DNA, 97 bp, synthetic construct
  source 1..97: mol_type = other DNA
catatggcccacatcgttatggtggatgcctacaagccaactaaaggttcaggaacagcaggtggtgggtcaggttccctgtctgttcctggtcccg

SEQ ID NO: 32 - DNA, 68 bp, synthetic construct
  source 1..68: mol_type = other DNA
catatggcccacatcgttatggtggatgcctacaagccaactaaagctagcaccggcaaaaatggtcg

SEQ ID NO: 33 - DNA, 33 bp, synthetic construct
  source 1..33: mol_type = other DNA
ggatccttaagcttccatcagcagcagttccgg

SEQ ID NO: 34 - DNA, 61 bp, synthetic construct
  source 1..61: mol_type = other DNA
gctagcggttcaggaacagcaggtggtgggtcaggttccccgcagcaagtcaaagacattc

SEQ ID NO: 35 - DNA, 40 bp, synthetic construct
  source 1..40: mol_type = other DNA
ggatccttatttttcgacacggtcaatttctttttgcagg

SEQ ID NO: 36 - protein, 279 aa, synthetic construct
ASTGKNGRVLKEDIDAFLAGGAKPGPAAAEEKAAPAAAKPATTEGEFPETREKMSGIRRAIAKAMVHSKHTAPHVTLMDEADVTKLVAHRKKFKAIAAEKGIKLTFLPYVVKALVSALREYPVLNTSIDDETEEIIQKHYYNIGIAADTDRGLLVPVIKHADRKPIFALAQEINELAEKARDGKLTPGEMKGASCTITNIGSAGGQWFTPVINHPEVAILGIGRIAEKPIVRDGEIVAAPMLALSLSFDHRMIDGATAQKALNHIKRLLSDPELLLMEA

SEQ ID NO: 37 - protein, 273 aa, synthetic construct
ASVLKEDIDAFLAGGAKPGPAAAEEKAAPAAAKPATTEGEFPETREKMSGIRRAIAKAMVHSKHTAPHVTLMDEADVTKLVAHRKKFKAIAAEKGIKLTFLPYVVKALVSALREYPVLNTSIDDETEEIIQKHYYNIGIAADTDRGLLVPVIKHADRKPIFALAQEINELAEKARDGKLTPGEMKGASCTITNIGSAGGQWFTPVINHPEVAILGIGRIAEKPIVRDGEIVAAPMLALSLSFDHRMIDGATAQKALNHIKRLLSDPELLLMEA

SEQ ID NO: 38 - DNA, 34 bp, synthetic construct
  source 1..34: mol_type = other DNA
catatgtcgtactaccatcaccatcaccatcacg

SEQ ID NO: 39 - DNA, 36 bp, synthetic construct
  source 1..36: mol_type = other DNA
gctagcaatatgagcgtcacctttagttgctttgcc

SEQ ID NO: 40 - DNA, 20 bp, synthetic construct
  source 1..20: mol_type = other DNA
tccatgacgttcctgacgtt

SEQ ID NO: 41 - protein, 568 aa, Influenza A virus
MEKIVLLFAIVSLVKSDQICIGYHANNSTEQVDTIMEKNVTVTHAQDILEKKHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQILSIYSTVASSLALAIMVAGLSLWMCSNGSLQCRICI

SEQ ID NO: 42 - DNA, 1977 bp, synthetic construct
  source 1..1977: mol_type = other DNA
atgtacaggatgcaactcctgtcttgcattgcactaagtcttgcacttgtcacaaacagtcatcatcaccatcatcaccatcaccattcaggaggaggatcaggaggaggaatcgagggaagggatagtgctacccatattaaattctcaaaacgtgatgaggacggcaaagagttagctggtgcaactatggagttgcgtgattcatctggtaaaactattagtacatggatttcagatggacaagtgaaagatttctacctgtatccaggaaaatatacatttgtcgaaaccgcagcaccagacggttatgaggtagcaactgctattacctttacagttaatgagcaaggtcaggttactgtaaatggcaaagcaactaaaggtgacgctcatattgctagcggttcaggaacagcaggtggtgggtcaggttccgatcagatttgcattggttaccatgcaaacaactcgacagagcaggttgacacaataatggaaaagaacgttactgttacacatgcccaagacatactggaaaagacacacaatgggaagctctgcgatctagatggagtgaagcctctaattttgagagattgtagtgtagctggatggctcctcggaaacccaatgtgtgacgaattcatcaatgtgccggaatggtcttacatagtggagaaggccaatccagtcaatgacctctgttacccaggggatttcaatgactatgaagaattgaaacacctattgagcagaataaaccattttgagaaaattcagatcatccccaaaagttcttggtccagtcatgaagcctcattgggggtgagctcagcatgtccataccagggaaagtcctcctttttcagaaatgtggtatggcttatcaaaaagaacagtacatacccaacaataaagaggagctacaataataccaaccaagaagatcttttggtactgtgggggattcaccatcctaatgatgcggcagagcagacaaagctctatcaaaacccaaccacctatatttccgttgggacatcaacactaaaccagagattggtaccaagaatagctactagatccaaagtaaacgggcaaagtggaaggatggagttcttctggacaattttaaaaccgaatgatgcaatcaacttcgagagtaatggaaatttcattgctccagaatatgcatacaaaattgtcaagaaaggggactcaacaattatgaaaagtgaattggaatatggtaactgcaacaccaagtgtcaaactccaatgggggcgataaactctagcatgccattccacaatatacaccctctcaccatcggggaatgccccaaatatgtgaaatcaaacagattagtccttgcgactgggctcagaaatagccctcaacgagagacgcgaggattatttggagctatagcaggttttatagagggaggatggcagggaatggtagatggttggtatgggtaccaccatagcaacgagcaggggagtgggtacgctgcagacaaagaatccactcaaaaggcaatagatggagtcaccaataaggtcaactcgattattgacaaaatgaacactcagtttgaggccgttggaagggaatttaacaacttagaaaggagaatagagaatttaaacaagaagatggaagacgggttcctagatgtctggacttataatgctgaacttctagttctcatggaaaacgagagaactctagactttcatgactcaaatgtcaagaacctttacgacaaggtccgactacagcttagggataatgcaaaggagctgggtaacggttgtttcgagttctatcataaatgtgataatgaatgtatggaaagtgtaagaaacggaacgtatgactacccgcagtattcagaagaagcaagactaaaaagagaggaaataagtggagtaaaattggaatcaataggaatttaccaaatataa

SEQ ID NO: 43 - protein, 658 aa, synthetic construct
MYRMQLLSCIALSLALVTNSHHHHHHHHHSGGGSGGGIEGRDSATHIKFSKRDEDGKELAGATMELRDSSGKTISTWISDGQVKDFYLYPGKYTFVETAAPDGYEVATAITFTVNEQGQVTVNGKATKGDAHIASGSGTAGGGSGSDQICIGYHANNSTEQVDTIMEKNVTVTHAQDILEKTHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRETRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQI

SEQ ID NO: 44 - protein, 256 aa, synthetic construct
PAPAAAEEKAAPAAAKPATTEGEFPETREKMSGIRRAIAKAMVHSKHTAPHVTLMDEADVTKLVAHRKKFKAIAAEKGIKLTFLPYVVKALVSALREYPVLNTSIDDETEEIIQKHYYNIGIAADTDRGLLVPVIKHADRKPIFALAQEINELAEKARDGKLTPGEMKGASCTITNIGSAGGQWFTPVINHPEVAILGIGRIAEKPIVRDGEIVAAPMLALSLSFDHRMIDGATAQKALNHIKRLLSDPELLLMEA

SEQ ID NO: 45 - protein, 260 aa, synthetic construct
MLSVPAPAAAEEKAAPAAAKPATTEGEFPETREKMSGIRRAIAKAMVHSKHTAPHVTLMDEADVTKLVAHRKKFKAIAAEKGIKLTFLPYVVKALVSALREYPVLNTSIDDETEEIIQKHYYNIGIAADTDRGLLVPVIKHADRKPIFALAQEINELAEKARDGKLTPGEMKGASCTITNIGSAGGQWFTPVINHPEVAILGIGRIAEKPIVRDGEIVAAPMLALSLSFDHRMIDGATAQKALNHIKRLLSDPELLLMEA